(19) United States
(12) Patent Application Publication
(10) Pub. No.: US 2026/0240590 A1
(43) Pub. Date: Aug. 20, 2026

(54) ULTRASONIC TREATMENT TOOL

(71) Applicant: OLYMPUS MEDICAL SYSTEMS CORP., Tokyo (JP)

(72) Inventor: Tsunetaka AKAGANE, Tokyo (JP)

(73) Assignee: OLYMPUS MEDICAL SYSTEMS CORP., Tokyo (JP)

(21) Appl. No.: 19/646,890

(22) Filed: Apr. 14, 2026

Related U.S. Application Data
(62) Division of application No. 18/109,938, filed on Feb. 15, 2023, now abandoned.
(60) Provisional application No. 63/311,085, filed on Feb. 17, 2022.
(60) Provisional application No. 63/311,068, filed on Feb. 17, 2022.

(51) Int. Cl. A61B 18/14; A61B 18/00

(52) U.S. Cl. CPC A61B 18/1445; A61B 2018/00077; A61B 2018/00083; A61B 2018/00136; A61B 2018/00601; A61B 2018/0063; A61B 2018/00994; A61B 2018/1452; A61B 2018/1467

(57) ABSTRACT

An ultrasonic treatment tool comprises: a blade including a treatment portion at a distal side of the blade, the blade configured to transmit an ultrasonic vibration from a proximal side of the blade to the treatment portion and the treatment portion configured to treat a body tissue, a grasper movable relative to the treatment portion between an open position and a closed position to grasp the body tissue between the grasper and the treatment portion, a coating provided on the grasper, the coating is formed by a first resin, when the grasper is in the closed position, the coating contacts the treatment portion.

### CROSS-REFERENCE TO RELATED APPLICATION

This application is a divisional application of U.S. Application No. 18/109,938 filed on February 15, 2023, and is based on and claims priority under 35 U.S.C. §119 to U.S. Provisional Application Nos. 63/311,068 and 63/311,085, each of which was filed on February 17, 2022. The entire contents of each of these applications are incorporated herein by reference.

### BACKGROUND

### 1. Technical Field

The present disclosure relates to ultrasonic treatment tools.

### 2. Related Art

An ultrasonic treatment tool, which is for treatment of a region to be treated (hereinafter, referred to as a treatment target) in body tissue by application of ultrasonic energy to the treatment target, has been known conventionally (see, for example, International Publication WO No. 2018/011918).

The ultrasonic treatment tool described in International Publication WO No. 2018/011918 includes a rod member, a holder member, and a pad member (hereinafter, referred to as the pad), which will be described below.

The rod member has, at a distal end of the rod member, a treatment portion for the treatment of the treatment target, and transmits ultrasonic vibration to the treatment portion from a proximal end of the rod member.

The holder member is opened and closed, relatively to the treatment portion.

The pad is a structure formed of a resin material and is attached to the holder member. The pad grasps the treatment target between the pad and the treatment portion.

### SUMMARY

In some embodiments, an ultrasonic treatment tool comprises: a blade including a treatment portion at a distal side of the blade, the blade configured to transmit an ultrasonic vibration from a proximal side of the blade to the treatment portion and the treatment portion configured to treat a body tissue, a grasper movable relative to the treatment portion between an open position and a closed position to grasp the body tissue between the grasper and the treatment portion, a coating provided on the grasper, the coating is formed by a first resin, when the grasper is in the closed position, the coating contacts the treatment portion.

In some embodiments, an ultrasonic treatment tool comprises: a blade including a treatment portion at a distal side of the blade, the blade configured to transmit an ultrasonic vibration from a proximal side of the blade to the treatment portion and the treatment portion configured to treat a body tissue, a grasper movable relative to the treatment portion between an open position and a closed position to grasp the body tissue between the grasper and the treatment portion, the grasper includes: a plurality of pads extending in a longitudinal direction of the grasper, the plurality of pads contacting the treatment portion when the grasper is in the closed position, an electrode between the plurality of pads and configured to flow a high frequency current between the electrode and the treatment portion when the grasper is in the closed position, a pair of insulation members extending across the plurality of pads and the electrode.

In some embodiments, an ultrasonic treatment tool comprises: a blade including a treatment portion at a distal side of the blade, the blade configured to transmit an ultrasonic vibration from a proximal side of the blade to the treatment portion and the treatment portion configured to treat a body tissue, a grasper movable relative to the treatment portion between an open position and a closed position to grasp the body tissue between the grasper and the treatment portion, a pad structure provided on the grasper, the pad structure contacts the treatment portion when the grasper is in the closed position, the pad structure includes: a first pad including a first resin having a first average molecular weight of 10,000,000 or more, a second pad including a second resin having a second average molecular weight less than the first average molecular weight, a first surface of the first pad is oriented toward a surface of the grasper and the second pad is on a second surface of the first pad.

The above and other features, advantages and technical and industrial significance of this disclosure will be better understood by reading the following detailed description of embodiments of the disclosure, when considered in connection with the accompanying drawings.

### BRIEF DESCRIPTION OF THE DRAWINGS

FIG. 1 is a diagram illustrating a treatment system according to a first embodiment.

FIG. 2 is a diagram illustrating a distal end portion of an ultrasonic treatment tool.

FIG. 3 is a diagram illustrating a configuration of a jaw and a vibration transmission member.

FIG. 4 is a diagram illustrating a modified example 1-1 of the first embodiment.

FIG. 5 is a diagram illustrating a modified example 1-2 of the first embodiment.

FIG. 6 is a diagram illustrating a modified example 1-3 of the first embodiment.

FIG. 7 is a diagram illustrating a modified example 1-4 of the first embodiment.

FIG. 8 is a diagram illustrating a modified example 1-5 of the first embodiment.

FIG. 9 is a diagram illustrating a modified example 1-6 of the first embodiment.

FIG. 10 is a diagram illustrating a modified example 1-7 of the first embodiment.

FIG. 11 is a diagram illustrating the modified example 1-7 of the first embodiment.

FIG. 12 is a diagram illustrating the modified example 1-7 of the first embodiment.

FIG. 13 is a diagram illustrating a modified example 1-8 of the first embodiment.

FIG. 14 is a diagram illustrating a modified example 1-9 of the first embodiment.

FIG. 15 is a diagram illustrating a configuration of pads and electrodes according to a second embodiment.

FIG. 16 is a diagram illustrating the configuration of the pads and the electrodes according to the second embodiment.

FIG. 17 is a diagram illustrating the configuration of the pads and the electrodes according to the second embodiment.

FIG. 18 is a diagram illustrating a modified example 2-1 of the second embodiment.

FIG. 19 is a diagram illustrating a modified example 2-2 of the second embodiment.

FIG. 20 is a diagram illustrating a modified example 2-3 of the second embodiment.

FIG. 21 is a diagram illustrating a modified example 2-4 of the second embodiment.

FIG. 22 is a diagram illustrating a modified example 2-5 of the second embodiment.

FIG. 23 is a diagram illustrating the modified example 2-5 of the second embodiment.

FIG. 24 is a diagram illustrating a modified example 2-6 of the second embodiment.

FIG. 25 is a diagram illustrating a modified example 3-1 of the first or second embodiment.

FIG. 26 is a diagram illustrating a modified example 3-2 of the first or second embodiment.

FIG. 27 is a diagram illustrating a modified example 3-3 of the first or second embodiment.

FIG. 28 is a diagram illustrating a modified example 3-3 of the first or second embodiment.

FIG. 29 is a diagram illustrating a modified example 3-4 of the first or second embodiment.

FIG. 30 is a diagram illustrating a modified example 3-5 of the first or second embodiment.

### DETAILED DESCRIPTION

Modes for implementing the disclosure (hereinafter, embodiments) will be described hereinafter while reference is made to the drawings. The disclosure is not limited by the embodiments described hereinafter. Furthermore, any portions that are the same will be assigned with the same reference sign, throughout the drawings.

### First Embodiment

### Schematic Configuration of Treatment System

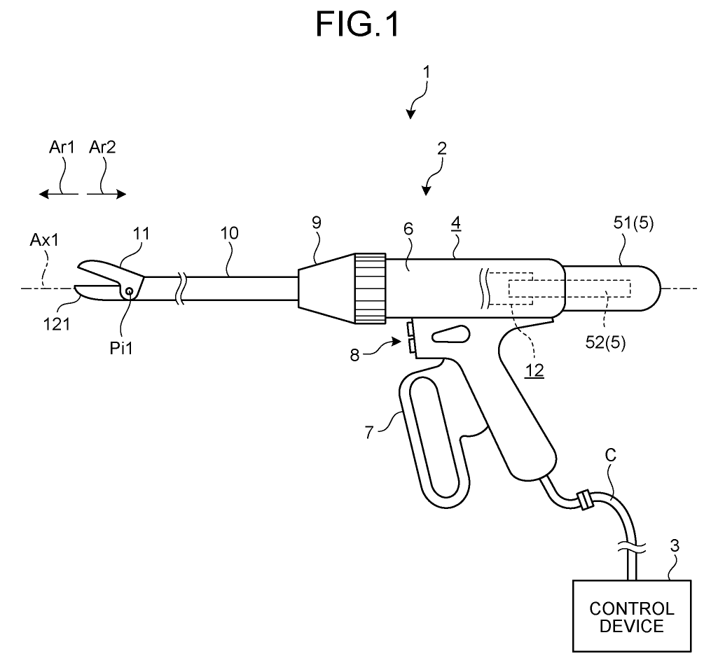

FIG. 1 is a diagram illustrating a treatment system 1 according to a first embodiment.

The treatment system 1 is for treatment of a part to be treated in body tissue (hereinafter, referred to as a treatment target) by application of treatment energy to the treatment target. The treatment energy according to this first embodiment is ultrasonic energy and high frequency energy. Furthermore, treatment that is enabled by the treatment system 1 according to the first embodiment is, for example, coagulation (sealing) of a treatment target or incision of a treatment target. Furthermore, the coagulation and the incision may be performed at the same time. The treatment energy to be applied to a treatment target is not necessarily both ultrasonic energy and high frequency energy and may be just ultrasonic energy. This treatment system 1 includes, as illustrated in FIG. 1, an ultrasonic treatment tool 2 and a control device 3.

### Configuration of Ultrasonic Treatment Tool

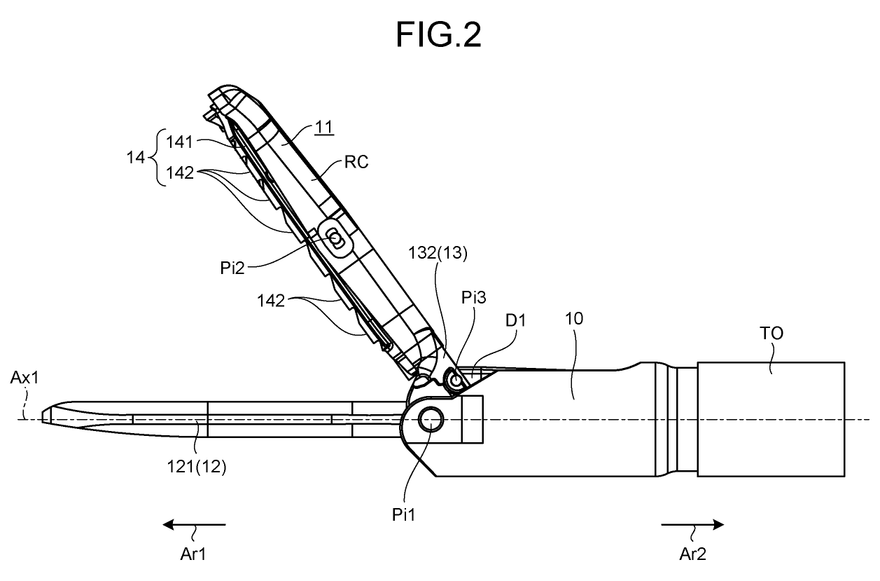

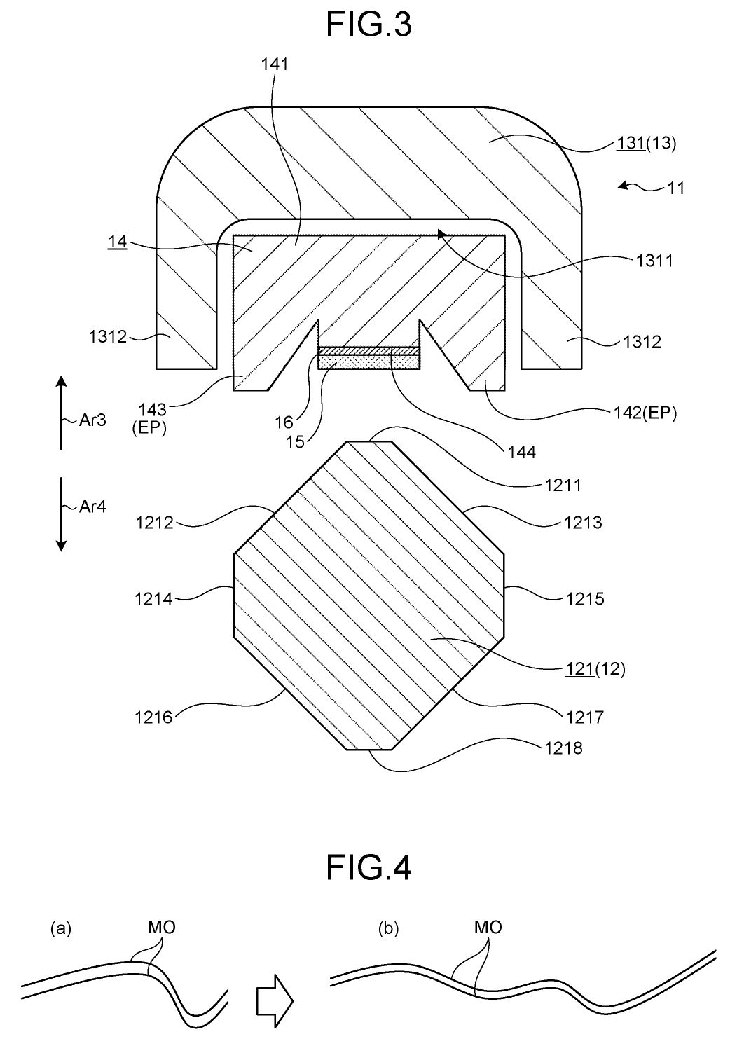

Hereinafter, one of directions along a central axis Ax1 (FIG. 1) of a sheath 10 will be referred to as a distal direction Ar1 and the other direction will be referred to as a proximal direction Ar2. Furthermore, a "width direction" referred to hereinafter means a direction orthogonal to: the central axis Ax1; and an opening and closing direction of a jaw 11 relative to a treatment portion 121, the direction being orthogonal to the plane of paper of FIG. 1 and FIG. 2 and lateral in FIG. 3.

FIG. 2 is a diagram illustrating a configuration of a distal end portion of the ultrasonic treatment tool 2. Specifically, FIG. 2 is a diagram of the distal end portion of the ultrasonic treatment tool 2 as viewed along the width direction.

The ultrasonic treatment tool 2 is a treatment tool for treatment of a treatment target by application of ultrasonic energy and high frequency energy to the treatment target. This ultrasonic treatment tool 2 includes, as illustrated in FIG. 1, a handpiece 4 and an ultrasonic transducer unit 5.

The handpiece 4 includes, as illustrated in FIG. 1 and FIG. 2, a holding case 6 (FIG. 1), an operation handle 7 (FIG. 1), switches 8 (FIG. 1), a rotation knob 9 (FIG. 1), the sheath 10, the jaw 11, and a vibration transmission member 12 (blade).

The holding case 6 supports the whole ultrasonic treatment tool 2.

The operation handle 7 is movably attached to the holding case 6 and receives an opening operation or a closing operation from an operator, such as an operating surgeon.

The switches 8 are provided in a state of being exposed to the exterior of the holding case 6 and receive treatment operations from an operator, such as an operating surgeon.

The rotation knob 9 has an approximately cylindrical shape coaxial with the central axis Ax1 and is provided near an end of the holding case 6, the end being in the distal direction Ar1. The rotation knob 9 receives a rotating operation from an operator, such as an operating surgeon. The rotating operation causes the rotation knob 9 to rotate about the central axis Ax1, relatively to the holding case 6. Furthermore, rotation of the rotation knob 9 causes the sheath 10, the jaw 11, and the vibration transmission member 12 to rotate about the central axis Ax1.

The sheath 10 is a cylindrical pipe formed of an electrically conducting material, such as metal.

A first pin Pi1 (FIG. 1 and FIG. 2) that extends in the width direction and is cylindrical is fixed to an end portion of the sheath 10, the end portion being in the distal direction Ar1.

An outer peripheral surface of the sheath 10 is covered with an electrically insulating outer tube TO (FIG. 2). Furthermore, an inner peripheral surface of the sheath 10 is covered with an electrically insulating inner tube (not illustrated in the drawings).

FIG. 3 is a diagram illustrating a configuration of the jaw 11 and the vibration transmission member 12. Specifically, FIG. 3 is a cross section of the jaw 11 and the vibration transmission member 12 cut along a plane orthogonal to the central axis Ax1. For convenience of explanation, illustration of a cover RC has been omitted, in FIG. 3.

In explaining a configuration of the jaw 11 hereinafter, a direction separating from the treatment portion 121 will be referred to as a rearward direction Ar3 and a direction approaching the treatment portion 121 will be referred to as a treatment portion direction Ar4.

The jaw 11 corresponds to a grasper. This jaw 11 is configured to be rotatable about a central axis of the first pin Pi1 (an axis along a direction orthogonal to the plane of paper of FIG. 1 or FIG. 2) by being pivotally supported about the first pin Pi1 at the end portion of the sheath 10, the end portion being in the distal direction Ar1. By rotating about the central axis of the first pin Pi1, the jaw 11 is opened or closed relatively to the treatment portion 121 provided at an end portion of the vibration transmission member 12, the end portion being in the distal direction Ar1. By the jaw 11 being closed relatively to the treatment portion 121, a treatment target is grasped between the jaw 11 and the treatment portion 121. The jaw 11 includes, as illustrated in FIG. 3, an arm 13 and a wiper jaw 14.

The arm 13 is formed of an electrically conducting material. This arm 13 is, as illustrated in FIG. 2 and FIG. 3, a member including an arm main body 131 (FIG. 3) and a bearing portion 132 (FIG. 2) that have been formed integrally with each other.

The arm main body 131 is formed of an approximately platy body that is elongated. In this first embodiment, a longitudinal direction of the arm main body 131 is a direction along a curve directed to the left in the distal direction Ar1 when viewed from the proximal direction Ar2, in a state where the jaw 11 is positioned above the treatment portion 121.

As illustrated in FIG. 3, a recessed portion 1311 is provided on a surface of this arm main body 131, the surface being in the treatment portion direction Ar4, the recessed portion 1311 extending from a proximal end of the arm main body 131 along the longitudinal direction in the distal direction Ar1.

A second pin Pi2 (FIG. 2) that extends in the width direction and is cylindrical is fixed by welding at a position that is at an approximate center of a longitudinal length of the jaw 11, the position being in side wall portions 1312 at both ends of a width of the arm main body 131, the side wall portions 1312 forming the recessed portion 1311.

Furthermore, the cover RC (FIG. 2 and FIG. 3) formed of electrically insulating resin is integrally formed on a surface of the arm main body 131, the surface being in the rearward direction Ar3, in a state where the surface in the rearward direction Ar3 is coated with the cover RC. In this first embodiment, the cover RC is formed by insert molding for the arm main body 131, but the first embodiment is not limited to this example. For example, in another adoptable configuration, the cover RC is fixed to the arm main body 131 by snap fitting or use of a metal pin.

The bearing portion 132 is a portion provided at the proximal end of the arm main body 131 and pivotally supported on the sheath 10 by the first pin Pi1.

Furthermore, a third pin Pi3 (FIG. 2) is fixed to the bearing portion 132 by welding, the third pin Pi3 extending in the width direction and being cylindrical. This third pin Pi3 is connected to an opening and closing mechanism D1 (FIG. 2) inserted inside and through the sheath 10. In association with movement of the opening and closing mechanism D1 in the distal direction Ar1 or the proximal direction Ar2, the movement corresponding to an opening or closing operation on the operation handle 7 by an operator, such as an operating surgeon, the jaw 11 is rotated about the central axis of the first pin Pi1 to be opened or closed relatively to the treatment portion 121.

The wiper jaw 14 is formed of an electrically conducting material, such as stainless steel or a titanium alloy, and is attached to the arm 13. As illustrated in FIG. 3, this wiper jaw 14 includes a wiper jaw main body 141, plural first tooth portions 142, and plural second tooth portions 143.

The wiper jaw main body 141 is formed of an elongated platy body extending along the longitudinal direction of the arm main body 131. Furthermore, the wiper jaw main body 141 has an outer shape that is set to be approximately the same as an inner shape of the recessed portion 1311. Inside the recessed portion 1311, the second pin Pi2 penetrates the wiper jaw main body 141 along the width direction, and the wiper jaw main body 141 is pivotally supported on the arm 13 so that the wiper jaw main body 141 is able to swing about a central axis (an axis along the width direction) of the second pin Pi2. That is, enabling the wiper jaw 14 to swing about the central axis of the second pin Pi2 allows a position to be positioned at the approximate center of the longitudinal length of the jaw 11, and not somewhere in the proximal direction Ar2 in the jaw 11, the position being where the strongest force is applied to a treatment target when the treatment target is grasped between the jaw 11 and the treatment portion 121. Force is thereby applied substantially evenly to the treatment target grasped between the jaw 11 and the treatment portion 121.

The plural first tooth portions 142 each protrude in the treatment portion direction Ar4 from one end of a width of a surface of the wiper jaw main body 141, the surface being in the treatment portion direction Ar4, and are arranged in parallel in a longitudinal direction of the wiper jaw main body 141.

The plural second tooth portions 243 each protrude in the treatment portion direction Ar4 from the other end of the width of the surface of the wiper jaw main body 141, the surface being in the treatment portion direction Ar4, and are arranged in parallel in the longitudinal direction of the wiper jaw main body 141.

A center area 144 that is positioned between the plural first tooth portions 142 and the plural second tooth portions 143 and is at the center of the width of the surface of the wiper jaw main body 141, the surface being in the treatment portion direction Ar4, is formed of a flat surface, as illustrated in FIG. 3. A pad 15 is provided on the center area 144, as illustrated in FIG. 3.

A detailed configuration of the pad 15 will be described in a later section, "Configuration of Pad".

The vibration transmission member 12 has an elongated shape and is formed of an electrically conducting material. Furthermore, the vibration transmission member 12 is inserted inside and through the sheath 10 in a state where the treatment portion 121 protrudes outside the sheath 10, as illustrated in FIG. 2. Upon this insertion, an end portion of the vibration transmission member 12, the end portion being in the proximal direction Ar2, is mechanically connected to an ultrasonic transducer 52 included in the ultrasonic transducer unit 5, as illustrated in FIG. 1. The vibration transmission member 12 transmits ultrasonic vibration generated by the ultrasonic transducer unit 5 from the end portion of the vibration transmission member 12 to the treatment portion 121, the end portion being in the proximal direction Ar2. In this first embodiment, the ultrasonic vibration is longitudinal vibration that is vibration along the central axis Ax1.

In this first embodiment, similarly to the jaw 11, the treatment portion 121 extends along a curve directed to the left in the distal direction Ar1 when viewed from the proximal direction Ar2, in a state where the jaw 11 is positioned above the treatment portion 121. Furthermore, the treatment portion 121 has, as illustrated in FIG. 3, an approximately octagon-shaped cross section cut along a plane orthogonal to the central axis Ax1. The octagon shape of the cross section of the treatment portion 121 is just an example, and the cross section may have a circular shape or any other shape. For convenience of explanation, the cross section of the treatment portion 121 will be assumed to be octagon-shaped in the description hereinafter.

A flat surface of the treatment portion 121, the flat surface being positioned near the jaw 11, will hereinafter be referred to as a first surface 1211. Furthermore, surfaces connected to the first surface 1211 along a circumferential direction around a central axis of the treatment portion 121 will be referred to as a second surface 1212 and a third surface 1213. Furthermore, surfaces respectively connected to the second and third surfaces 1212 and 1213 along the circumferential direction around the central axis of the treatment portion 121 will be referred to as a fourth surface 1214 and a fifth surface 1215. Furthermore, surfaces respectively connected to the fourth and fifth surfaces 1214 and 1215 along the circumferential direction around the central axis of the treatment portion 121 will be referred to as a sixth surface 1216 and a seventh surface 1217. Furthermore, a surface positioned between the sixth and seventh surfaces 1216 and 1217 and opposite to the first surface 1211 will be referred to as an eighth surface 1218.

The ultrasonic transducer unit 5 includes, as illustrated in FIG. 1, a transducer (TD) case 51 and the ultrasonic transducer 52.

The TD case 51 supports the ultrasonic transducer 52 and is detachably connected to the holding case 6.

The ultrasonic transducer 52 generates ultrasonic vibration, under control by the control device 3. In this embodiment, the ultrasonic transducer 52 is a bolt-clamped Langevin transducer (BLT).

### Configuration of Control Device

The control device 3 integrally controls operation of the ultrasonic treatment tool 2 through an electric cable C (FIG. 1).

Specifically, the control device 3 detects a treatment operation on the switches 8 by an operator, such as an operating surgeon, through the electric cable C. In a case where the control device 3 has detected the treatment operation, the control device 3 applies, through the electric cable C, treatment energy to a treatment target grasped between the jaw 11 and the treatment portion 121. That is, the control device 3 implements treatment of the treatment target.

For example, in applying ultrasonic energy to a treatment target, the control device 3 supplies drive power to the ultrasonic transducer 52 through the electric cable C. The ultrasonic transducer 52 thereby generates longitudinal vibration (ultrasonic vibration) that is vibration along the central axis Ax. Furthermore, the treatment portion 121 is vibrated at a desired amplitude by the longitudinal vibration. The ultrasonic vibration is then applied from the treatment portion 121 to the treatment target grasped between the jaw 11 (the pad 15) and the treatment portion 121. In other words, ultrasonic energy is applied to the treatment target from the treatment portion 121.

Furthermore, for example, in applying high frequency energy to a treatment target, the control device 3 supplies high frequency power between the jaw 11 and the vibration transmission member 12 through the electric cable C. In response to supply of the high frequency power between the jaw 11 and the vibration transmission member 12, high frequency current flows to the treatment target positioned between the plural first and second tooth portions 142 and 143 and the treatment portion 121. In other words, high frequency energy is applied to the treatment target.

As described above, in this first embodiment, the plural first and second tooth portions 142 and 143 of the wiper jaw 14 function as electrodes EP (FIG. 3) that let high frequency current flow to the treatment target.

### Configuration of Pad

The pad 15 is formed of a resin material that is electrically insulating and biocompatible, for example, polytetrafluoroethylene (PTFE), and is a coating formed by a known coating process including coating. In this first embodiment, a surface of the pad 15 is a flat surface following the center area 144, the surface being in the treatment portion direction Ar4. The flat surfaces of the pad 15 and the first surface 1211 come into contact with each other when the jaw 11 is brought close to the treatment portion 121.

The pad 15 may have a thickness less than 1 mm. Furthermore, in a case where the pad 15 is formed by a known coating process including coating, forming a thick coating is expensive and the thickness of the pad 15 may be thus more 300 μm or smaller. Furthermore, to maintain electric insulation of the pad 15 even when the pad 15 is worn away, the thickness of the pad 15 may be 10 μm or larger.

In a case where the pad 15 that is a PTFE coating is formed on the center area 144 of the wiper jaw 14 formed of metal, a problem may be caused in sticking force between the pad 15 and the center area 144.

In this first embodiment, in consideration of the sticking force mentioned above, a plating layer 16 is provided between the center area 144 and the pad 15, as illustrated in FIG. 3.

The plating layer 16 is eutectic plating (eutectic alloy) including a resin component that is the same as the resin included in the pad 15. In this first embodiment, the plating layer 16 is Ni-PTFE plating. In a state where the pad 15 is provided on the plating layer 16, the resin component included in the plating layer 16 and the resin included in the pad 15 are fused together.

The plating layer 16 has a thickness of, for example, 1 μm or larger and 100 μm or smaller. Furthermore, a percentage of the resin component (PTFE) included in the plating layer 16 may be 30% or more for maintaining sticking force between the plating layer 16 and the pad 15. Furthermore, the percentage of the resin component (PTFE) included in the plating layer 16 may be 95% or less for maintaining strength of the plating layer 16.

The above described first embodiment has the following effects.

The pad 15 in the ultrasonic treatment tool 2 according to the first embodiment is formed of a coating including resin. Therefore, the pad 15 is able to be provided in a state of being firmly stuck to the center area 144 of the wiper jaw 14. That is, a structure is able to be obtained, the structure enabling reduction in thermal resistance between the center area 144 and the pad 15 and facilitating fast transmission of frictional heat to the wiper jaw 14 from the pad 15, the frictional heat having been generated in the pad 15 by application of ultrasonic vibration. Furthermore, because the pad 15 is able to be made very thin as compared to a conventional pad formed of a structure, the frictional heat is prevented from staying in the pad 15.

Therefore, the ultrasonic treatment tool 2 according to the first embodiment enables reduction of deterioration of the pad 15.

In particular, the thickness of the pad 15 is 10 μm or larger. Therefore, even if the pad 15 is worn away, electric insulation of the pad 15 is able to be maintained.

Furthermore, the thickness of the pad 15 is 300 μm or smaller. Therefore, the pad 15 is able to be formed inexpensively because forming a pad 15 formed as a thick coating of 1 mm, for example, is expensive.

Furthermore, the plating layer 16 described above is provided between the center area 144 and the pad 15 in the ultrasonic treatment tool 2 according to the first embodiment. Therefore, the sticking force of the pad 15 relative to the jaw 11 is able to be improved.

In particular, the percentage of the resin component (PTFE) included in the plating layer 16 is 30% or more. Therefore, the sticking force between the plating layer 16 and the pad 15 is able to be maintained.

Furthermore, the percentage of the resin component (PTFE) included in the plating layer 16 is 95% or less. Therefore, strength of the plating layer 16 is able to be maintained.

Modified Example 1-1

The pad 15 in the above described first embodiment may be formed of a resin material (PTFE) having an average molecular weight of 10,000,000 or more and having no side chain.

FIG. 4 is a diagram illustrating a modified example 1-1 of the first embodiment. Specifically, FIG. 4 is a diagram illustrating effects of the modified example 1-1.

The modified example 1-1 described above has the following effects, in addition to effects similar to those of the first embodiment described above.

In a case where a pad 15 is formed of a resin (PTFE) having an average molecular weight of 10,000,000 or less, as illustrated in (a) of FIG. 4, polymers MO of this PTFE are comparatively short in length and intermolecular force acting between the polymers MO is comparatively low. Therefore, such a pad 15 tends to be deteriorated by frictional heat generated by application of ultrasonic vibration.

By contrast, the pad 15 in the ultrasonic treatment tool 2 according to the modified example 1-1 includes a resin (PTFE) having an average molecular weight of 10,000,000 or more. Therefore, as illustrated in (b) of FIG. 4, polymers MO of the PTFE are long in length and intermolecular force acting between the polymers MO is comparatively high.

Therefore, the ultrasonic treatment tool 2 according to the modified example 1-1 enables reduction of deterioration of the pad 15 due to frictional heat generated by application of ultrasonic vibration.

In particular, the resin (PTFE) forming the pad 15 has no side chain. Therefore, close portions of the polymers MO of the PTFE are able to be increased, the close portions being close to each other, and intermolecular force acting between the polymers MO is able to be increased.

The pad 15 according to the modified example 1-1 is not necessarily a coating, and may be a structure configured to be fixed to the wiper jaw 14. A method of fixing such a pad 15 to the wiper jaw 14 may be, for example: a method of making claw portions protruding from the wiper jaw 14, and mechanically fixing the pad 15 to the wiper jaw 14 by engaging the pad 15 with the claw portions; or insert molding. In this case, the plating layer 16 is not required.

Modified Example 1-2

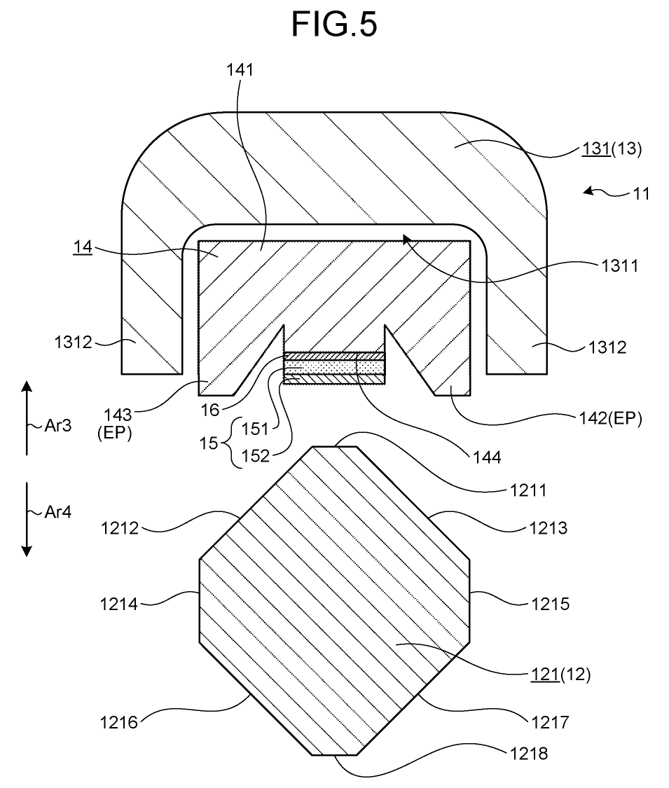

FIG. 5 is a diagram illustrating a modified example 1-2 of the first embodiment. Specifically, FIG. 5 a diagram corresponding to FIG. 3.

The pad 15 in the modified example 1-1 described above may include a first pad 151 and a second pad 152 layered over each other, like in the modified example 1-2 illustrated in FIG. 5.

The first pad 151 has a configuration similar to that of the pad 15 explained with respect to the modified example 1-1 described above.

The second pad 152 is formed of a resin material that is electrically insulating and biocompatible, for example, PTFE that is the same as that of the first pad 151, and is a coating provided on a surface of the first pad 151 by a known coating process including coating. In this modified example 1-2, the resin material (PTFE) forming the second pad 152 has an average molecular weight (for example, about 200,000) that is lower than that of the first pad 151 and the second pad 152 has a thickness dimension smaller than that of the first pad 151. Furthermore, a surface of the second pad 152 is a flat surface following the center area 144, the surface being in the treatment portion direction Ar4. The flat surfaces of the second pad 152 and the first surface 1211 come into contact with each other when the jaw 11 is brought close to the treatment portion 121.

The modified example 1-2 described above has the following effects, in addition to effects similar to those of the modified example 1-1 described above.

The PTFE forming the second pad 152 and having a lower molecular weight is high in crystallinity and thus has a small friction coefficient.

In this modified example 1-2, the second pad 152 is provided on the surface of the first pad 151. Therefore, providing the second pad 152 enables reduction of frictional heat that is generated in the pad 15 by application of ultrasonic vibration.

The pad 15 according to the modified example 1-2 is not necessarily a coating, and may be a structure configured to be fixed to the wiper jaw 14. A method of fixing such a pad 15 to the wiper jaw 14 may be, for example: a method of making claw portions protruding from the wiper jaw 14, and mechanically fixing the pad 15 to the wiper jaw 14 by engaging the pad 15 with the claw portions; or insert molding. In this case, the plating layer 16 is not required.

Modified Example 1-3

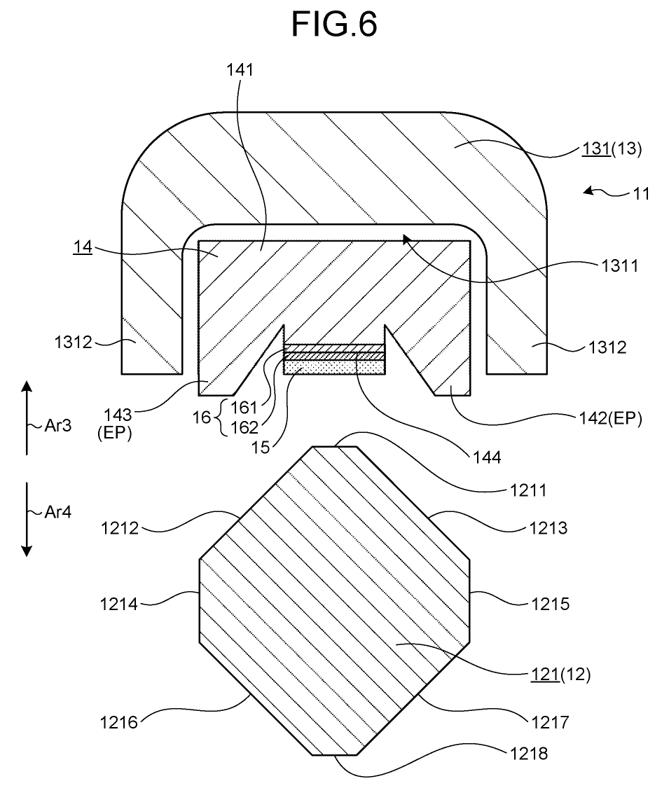

FIG. 6 is a diagram illustrating a modified example 1-3 of the first embodiment. Specifically, FIG. 6 a diagram corresponding to FIG. 3.

The plating layer 16 in the modified examples 1-1 and 1-2 described above may include plural (two in the modified example 1-3) layers (sub-layers) 161 and 162 layered over each other, like in the modified example 1-3 illustrated in FIG. 6. FIG. 6 illustrates, as an example, a case where the plating layer 16 according to the modified example 1-1 described above includes the layers 161 and 162. In this case, a percentage of a resin component (PTFE) in the layer 162 close to the pad 15 is higher than that in the layer 161 close to the center area 144. The percentages of the resin component (PTFE) included in the layers 161 and 162 may be 30% or more and 95% or less, as explained with respect to the first embodiment described above.

The modified example 1-3 described above has the following effects, in addition to effects similar to those of the modified examples 1-1 and 1-2 described above.

The plating layer 16 according to the modified example 1-3 includes the layers 161 and 162 described above. Therefore, sticking force between the center area 144 and the plating layer 16 and between the plating layer 16 and the pad 15 is able to be strengthened and as a result, sticking force of the pad 15 relative to the jaw 11 is able to be improved further.

Modified Example 1-4

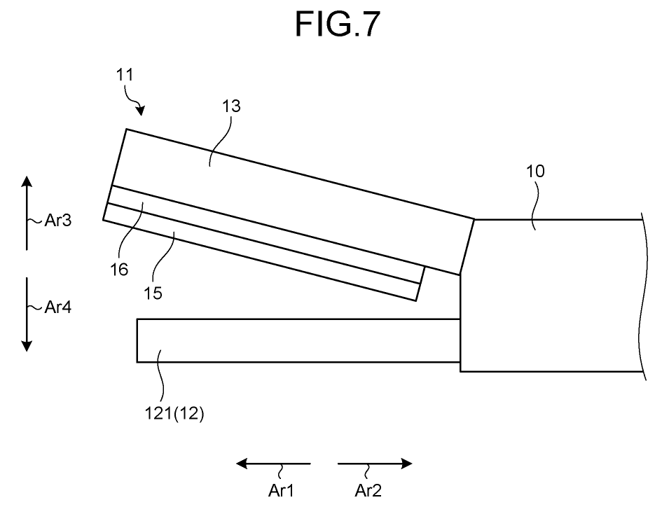

FIG. 7 is a diagram illustrating a modified example 1-4 of the first embodiment. Specifically, FIG. 7 is a diagram of the distal end portion of the ultrasonic treatment tool 2 as viewed along the width direction. For convenience of explanation, illustration of the cover RC has been omitted, in FIG. 7.

In the above described modified examples 1-1 to 1-3, the configuration adopted as the grasper includes the arm 13 and the wiper jaw 14 swingably attached to the arm 13, but the disclosure is not limited to this configuration. Any configuration without the wiper jaw 14 and capable of being opened and closed relatively to the treatment portion 121 may be adopted as the grasper instead. That is, the plating layer 16 and the pad 15 are provided on a surface of the arm 13, the surface being in the treatment portion direction Ar4. FIG. 7 illustrates, as an example, a case where the plating layer 16 and the pad 15 according to the modified example 1-1 described above are provided on the arm 13.

In a case where the structure of the modified example 1-4 described above is adopted, effects similar to those of the modified examples 1-1 to 1-3 described above are achieved also.

The pad 15 according to the modified example 1-4 is not necessarily a coating, and may be a structure configured to be fixed to the arm 13. A method of fixing such a pad 15 to the arm 13 may be, for example: a method of making claw portions protruding from the arm 13, and mechanically fixing the pad 15 to the arm 13 by engaging the pad 15 with the claw portions; or insert molding. In this case, the plating layer 16 is not required.

Modified Example 1-5

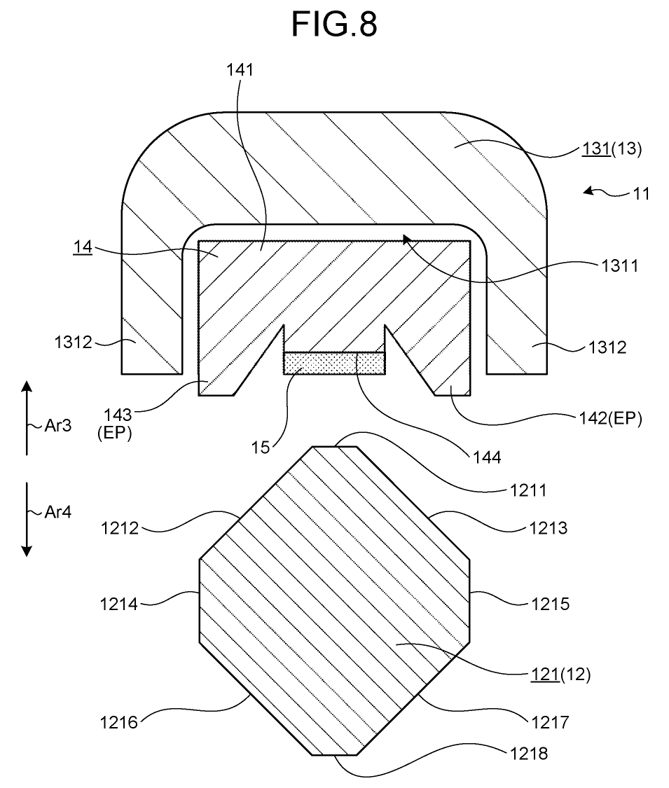

FIG. 8 is a diagram illustrating a modified example 1-5 of the first embodiment. Specifically, FIG. 8 a diagram corresponding to FIG. 3.

In the modified examples 1-1 to 1-4 described above, the pad 15 may be directly provided on the wiper jaw 14 or the arm 13 without provision of the plating layer 16. FIG. 8 illustrates, as an example, a configuration having the pad 15 directly provided on the wiper jaw 14 in the modified example 1-1 described above.

In a case where the structure of the modified example 1-5 described above is adopted, effects similar to those of the modified examples 1-1 to 1-4 described above are achieved also.

The resin forming the pad 15 in the above described modified examples 1-1 to 1-5 is not necessarily PTFE, and other resin, for example, other fluororesin, may be adopted instead.

Modified Example 1-6

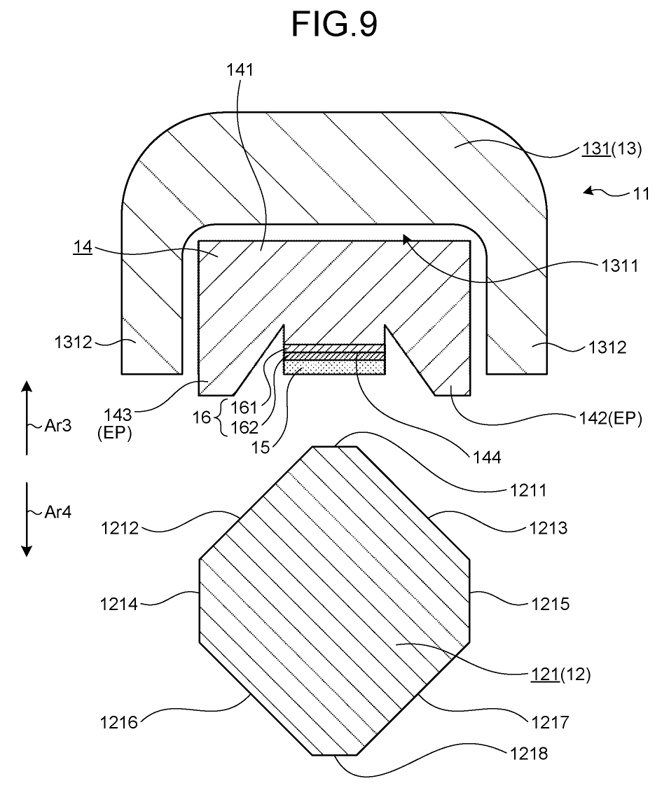

FIG. 9 is a diagram illustrating a modified example 1-6 of the first embodiment. Specifically, FIG. 9 a diagram corresponding to FIG. 3.

Similarly to the modified example 1-3 described above, the plating layer 16 in the above described first embodiment may include the plural (two in this modified example 1-6) layers 161 and 162 layered over each other like in the modified example 1-6 illustrated in FIG. 9. In this case, the percentage of the resin component (PTFE) in the layer 162 close to the pad 15 is higher than that in the layer 161 close to the center area 144. The percentages of the resin component (PTFE) included in the layers 161 and 162 may be 30% or more and 95% or less, as explained with respect to the first embodiment described above.

The above described modified example 1-6 has the following effects, in addition to effects similar to those of the first embodiment described above.

The plating layer 16 according to the modified example 1-6 includes the layer 161 and 162 described above. Therefore, sticking force between the center area 144 and the plating layer 16 and between the plating layer 16 and the pad 15 is able to be strengthened and as a result, sticking force of the pad 15 relative to the jaw 11 is able to be improved further.

Modified Example 1-7

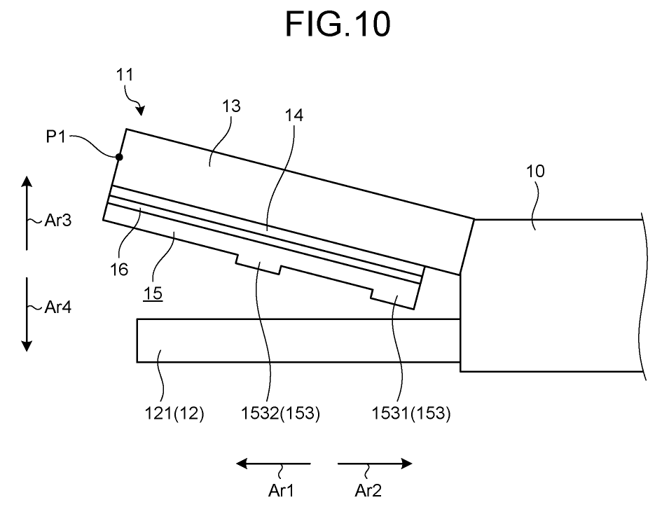

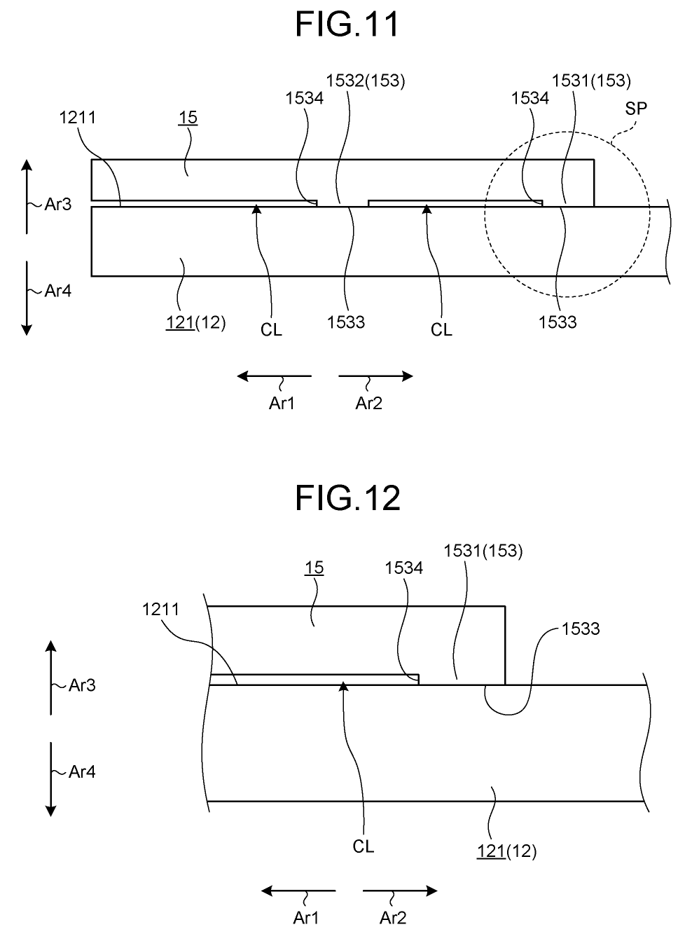

FIG. 10 to FIG. 12 are diagrams illustrating a modified example 1-7 of the first embodiment. Specifically, FIG. 10 is a diagram of the distal portion of the ultrasonic treatment tool 2 as viewed along the width direction. For convenience of explanation, illustration of the cover RC has been omitted, in FIG. 10. FIG. 11 is a diagram of a state where the pad 15 and the treatment portion 121 (the first surface 1211) are in contact with each other, as viewed along the width direction. FIG. 12 is an enlarged diagram of a region SP illustrated in FIG. 11.

A shape having projection portions 153 projecting in the treatment portion direction Ar4 from the surface of the pad 15, the surface being in the treatment portion direction Ar4, may be adopted as a shape of the surface in the above described first embodiment, like in the modified example 1-7 illustrated in FIG. 10 to FIG. 12.

The number of the projection portions 153 in this modified example 1-7 is two, as illustrated in FIG. 10 and FIG. 11. These two projection portions 153 are provided at positions separate from an antinode position P1 (FIG. 10) of ultrasonic vibration transmitted to the treatment portion 121. This antinode position P1 is a position of a distal end of the treatment portion 121.

Specifically, one projection portion 1531 of the two projection portions 153 is provided on an end portion of the surface of the pad 15, the surface being in the treatment portion direction Ar4, the end portion being in the proximal direction Ar2. Furthermore, the other projection portion 1532 is provided at a position on the surface of the pad 15, the surface being in the treatment portion direction Ar4, the position being at an approximate center of the longitudinal length of the jaw 11. That is, the position of the projection portion 1532 along the longitudinal direction of the jaw 11 is approximately the same as that of the second pin Pi2.

Length dimensions, along the width direction, of these two projection portions 153 are approximately the same as the length dimension, along the width direction, of the pad 15. Furthermore, tips 1533 of the two projection portions 153 each have a flat surface, as illustrated in FIG. 11. The flat surfaces of the two projection portions 153 and the first surface 1211 come into contact with each other when the jaw 11 is brought close to the treatment portion 121. In this state, a predetermined clearance CL (FIG. 11 and FIG. 12) is provided between an area of the surface of the pad 15 and the first surface 1211, the area being other than the two projection portions 153, the surface being in the treatment portion direction Ar4. That is, part of the clearance CL is provided between a portion of the pad 15 and the treatment portion 121, the portion being in the distal direction Ar1. Furthermore, side surfaces 1534 of the two projection portions 153 are each a surface orthogonal to the tips 1533, as illustrated in FIG. 12.

The projection portions 153 each may have a height dimension (a thickness dimension of the clearance CL) of a few micrometers (μm) or larger and 100 μm or smaller. Furthermore, as a result of, through simulation, setting the pad 15 at 200°C, with the clearance CL having a thickness dimension of 50 μm, and measuring a thickness dimension of the clearance CL after softening of the pad 15, the thickness dimension was found to be about 20 μm. In view of this simulation result, the height dimension of the projection portions 153 (the thickness dimension of the clearance CL) may be more 30 μm or larger.

The above described modified example 1-7 has the following effects, in addition to effects similar to those of the first embodiment described above.

PTFE that is a resin component is known to have a characteristic of having a lower friction coefficient when perpendicular force to PTFE is increased.

The pad 15 according to this modified example 1-7 has the projection portions 153 that come into contact with the treatment portion 121 when the jaw 11 is brought close to the treatment portion 121. Therefore, reducing the contact area over which the pad 15 is in contact with the treatment portion 121 increases pressure on the contacting portions (the projection portions 153) and as a result, the friction coefficient is decreased. Therefore, frictional heat generated in the pad 15 by application of ultrasonic vibration is able to be reduced and deterioration of the pad 15 is able to be reduced effectively.

In particular, the projection portions 153 are provided at the positions separate from the antinode position P1 where the amplitude of ultrasonic vibration transmitted to the treatment portion 121 reaches a maximum. Therefore, friction between the projection portions 153 and the treatment portion 121 is able to be reduced and frictional heat generated in the pad 15 is able to be reduced further.

In a case where the side surfaces 1534 intersect the tips 1533 at an obtuse angle, due to formation of a portion that comes into contact with the treatment portion 121 inadequately, setting the friction coefficient of the pad 15 small becomes difficult.

The side surfaces 1534 of the projection portions 153 of the pad 15 according to this modified example 1-7 are surfaces orthogonal to the tips 1533. Therefore, the friction coefficient of the pad 15 is able to be set small.

The pad 15 according to the modified example 1-7 is not necessarily a coating, and may be formed of a structure configured to be fixed to the wiper jaw 14. A method of fixing such a pad 15 to the wiper jaw 14 may be, for example: a method of making claw portions protruding from the wiper jaw 14, and mechanically fixing the pad 15 to the wiper jaw 14 by engaging the pad 15 with the claw portions; or insert molding. In this case, the plating layer 16 is not required.

Modified Example 1-8

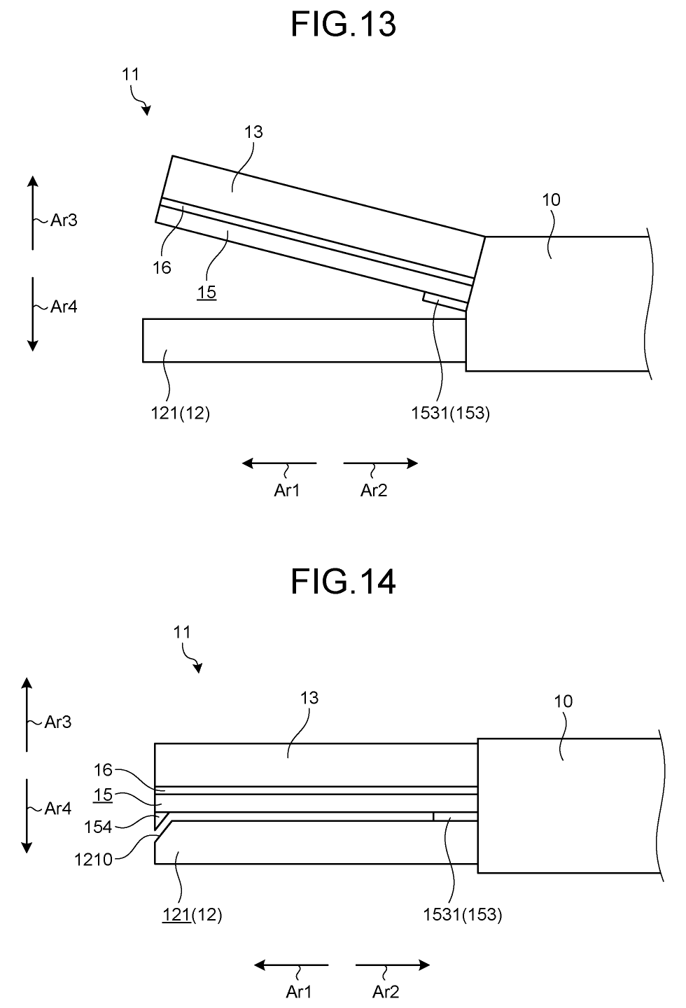

FIG. 13 is a diagram illustrating a modified example 1-8 of the first embodiment. Specifically, FIG. 13 is a diagram corresponding to FIG. 10.

The configuration including the arm 13 and the wiper jaw 14 swingably attached to the arm 13 is adopted as the grasper in the first embodiment and modified examples 1-6 and 1-7 described above, but the disclosure is not limited to this configuration. Any configuration without the wiper jaw 14 and capable of being opened and closed relatively to the treatment portion 121 may be adopted as the grasper instead. That is, the plating layer 16 and the pad 15 are provided on the surface of the arm 13, the surface being in the treatment portion direction Ar4. FIG. 13 illustrates, as an example, a case where the plating layer 16 and pad 15 according to the above described modified example 1-7 are provided on the surface of the arm 13, the surface being in the treatment portion direction Ar4. As to the projection portions 153 in this case, because the wiper jaw 14 that swings relatively to the arm 13 is not provided, the two projection portions 1531 and 1532 are not required to be provided and only one projection portion 1531 may thus be provided.

In a case where the structure of the modified example 1-8 described above is adopted, effects similar to those of the first embodiment and modified examples 1-6 and 1-7 described above are achieved also.

The pad 15 according to the modified example 1-8 is not necessarily a coating, and may be a structure configured to be fixed to the arm 13. A method of fixing this pad 15 to the arm 13 may be, for example: a method of making claw portions protruding from the arm 13, and mechanically fixing the pad 15 to the arm 13 by engaging the pad 15 with the claw portions; or insert molding. In this case, the plating layer 16 is not required.

Modified Example 1-9

FIG. 14 is a diagram illustrating a modified example 1-9 of the first embodiment. Specifically, FIG. 14 is a diagram corresponding to FIG. 13.

In the above described modified examples 1-7 and 1-8, a projection portion 154 that is approximately the same as the projection portion 153 may be provided at a distal end of the pad 15, to make it easier for body tissue to be grasped at an end portion of the jaw 11 and the treatment portion 121, the end portion being in the distal direction Ar1. FIG. 14 illustrates, as an example, a case where the projection portion 154 is provided at the distal end of the pad 15 in the above described modified example 1-8. In the case where the projection portion 154 is provided at the distal end of the pad 15, an inclined surface 1210 (FIG. 14) is provided at a corner portion of the distal end of the treatment portion 121 to prevent the projection portion 154 from coming into contact with the first surface 1211 when the jaw 11 is brought close to the treatment portion 121.

In a case where the structure of the modified example 1-9 described above is adopted, effects similar to those of the modified examples 1-7 and 1-8 described above are achieved also.

The pad 15 according to the modified example 1-9 is not necessarily a coating, and may be a structure configured to be fixed to the arm 13. A method of fixing such a pad 15 to the arm 13 may be, for example: a method of making claw portions protruding from the arm 13, and mechanically fixing the pad 15 to the arm 13 by engaging the pad 15 with the claw portions; or insert molding. In this case, the plating layer 16 is not required.

### Second Embodiment

A second embodiment will be described next.

In the following description, any component that is the same as that of the above described first embodiment will be assigned with the same reference sign, and detailed description thereof will be omitted or simplified.

In this second embodiment, pads 15 are modified in shape, and not first and second tooth portions 142 and 143 but part of a center area 144 of a wiper jaw 14 functions as electrodes EP, in contrast to the first embodiment described above.

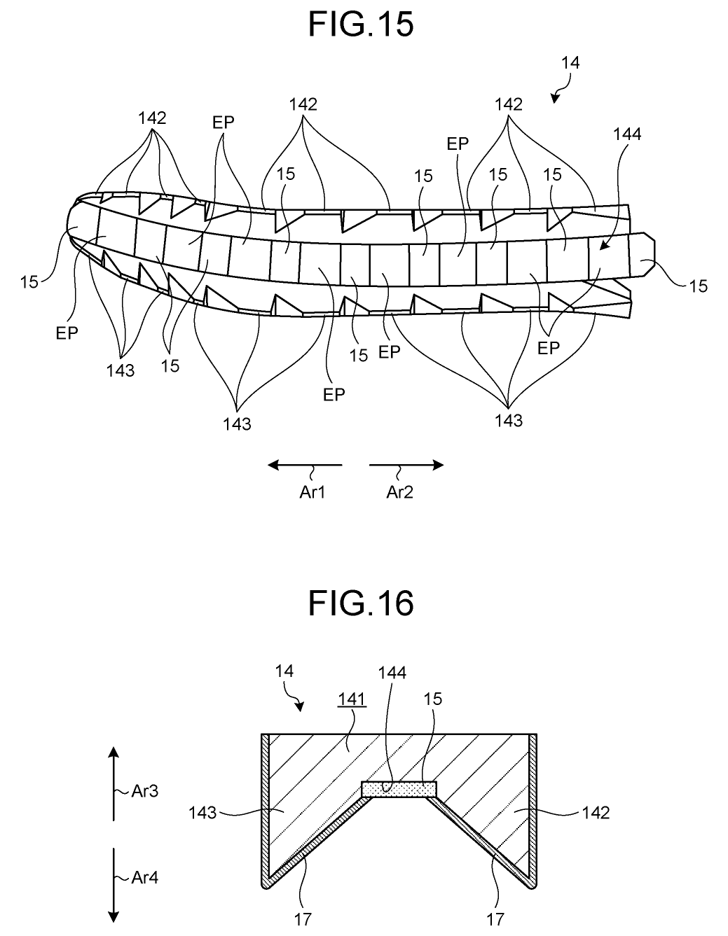

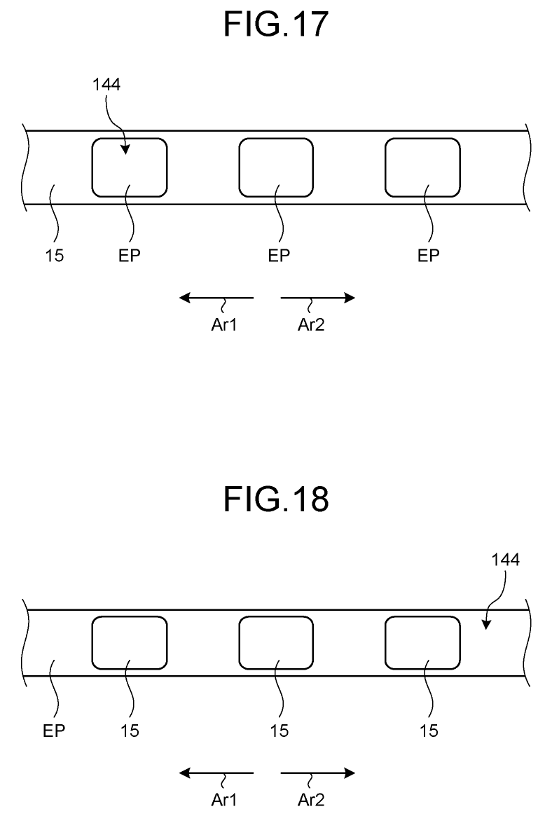

FIG. 15 to FIG. 17 are diagrams illustrating a configuration of the pads 15 and electrodes EP according to the second embodiment. Specifically, FIG. 15 is a diagram of the wiper jaw 14 as viewed from a treatment portion direction Ar4. For convenience of explanation, illustration of a first insulating portion 17 has been omitted, in FIG. 15. FIG. 16 is a cross section of the wiper jaw 14 cut along a plane orthogonal to a central axis Ax1. For convenience of explanation, illustration of a plating layer 16 has been omitted, in FIG. 16. FIG. 17 is an enlarged diagram of a part of the center area 144 in FIG. 15.

As illustrated in FIG. 15, the pads 15 are plurally provided on the center area 144, in a state where the pads 15 are arranged in parallel in a longitudinal direction of the wiper jaw 14.

Furthermore, surfaces of a pair of tooth portions 142 and 143 are covered with the first insulating portion 17 that is electrically insulating, as illustrated in FIG. 16. This first insulating portion 17 corresponds to an insulating portion. In this second embodiment, the first insulating portion 17 is a coating formed of electrically insulating resin. This resin is not particularly limited as long as the resin is electrically insulating. Furthermore, the first insulating portion 17 is not necessarily a coating, and a configuration, in which a cover formed of an electrically insulating material is attached to the wiper jaw 14 to cover the surfaces of the pair of tooth portions 142 and 143, may be adopted instead.

Accordingly, in this second embodiment, as illustrated in FIG. 15, regions including portions between adjacent ones of the pads 15 function as the electrodes EP, the regions being where none of the pads 15 is provided. When the jaw 11 is brought close to a treatment portion 121, only the plural pads 15 come into contact with a first surface 1211 and the plural electrodes EP do not come into contact with the first surface 1211.

A water-repellent coating may be formed on surfaces of the electrodes EP. Forming the water-repellant coating enables prevention of sticking of body tissue to the electrodes EP. Furthermore, the water-repellant coating may have a thickness of 1 μm or smaller, the thickness enabling high frequency current to flow.

In this second embodiment, the pads 15 are provided in a state of respectively surrounding the plural electrodes EP in a plan view as illustrated in FIG. 17. For example, after the plating layer 16 and the pads 15 are provided on the whole center area by a known coating process including coating, the electrodes EP may be provided by part of the plating layer 16 and pads 15 being peeled off. Furthermore, for example, the plating layer 16 and the pads 15 may be provided only in part of the center area 144 by a known coating process including coating.

The second embodiment described above has the following effects, in addition to effects similar to those of the first embodiment described above.

The pads 15 in the ultrasonic treatment tool 2 according to the second embodiment are plurally provided on the center area 144, in a state where the pads 15 are arranged in parallel in the longitudinal direction of the wiper jaw 14. Furthermore, the surfaces of the pairs of tooth portions 142 and 143 are covered with the first insulating portion 17 that is electrically insulating. The regions including the portions between adjacent ones of the pads 15 in the center area 144 function as the electrodes EP, the regions being where none of the pads 15 is provided.

Therefore, high frequency current flows between the center area 144 and the first surface 1211 and thermal spread outward along the width direction from the center area 144 is thus able to be reduced. Furthermore, high frequency current is allowed to flow to a portion where a treatment target is most compressed (between the center area 144 and the first surface 1211) and treatment performance is thus able to be improved.

The pads 15 according to this second embodiment are not necessarily coatings, and structures configured to be fixed to the wiper jaw 14 may be adopted as the pads 15 instead. A method of fixing such pads 15 to the wiper jaw 14 may be, for example: a method of making claw portions protruding from the wiper jaw 14, and mechanically fixing the pads 15 to the wiper jaw 14 by engaging the pads 15 with the claw portions; or insert molding. In this case, the plating layer 16 is not required.

Furthermore, in this second embodiment, a configuration without the wiper jaw 14 may be adopted as the grasper, similarly to the modified example 1-8 described above. In that case, the pads 15, the electrodes EP, and the first insulating portion 17 are provided on an arm 13 in an arrangement similar to the arrangement provided on the wiper jaw 14.

Modified Example 2-1

FIG. 18 is a diagram illustrating a modified example 2-1 of the second embodiment. Specifically, FIG. 18 is a diagram corresponding to FIG. 17.

The pads 15 in the above described second embodiment may be provided in a state of being surrounded by an electrode EP in a plan view, like in the modified example 2-1 illustrated in FIG. 18.

In a case where the above described structure according to the modified example 2-1 is adopted, effects similar to those of the second embodiment described above are achieved also.

The pads 15 according to the modified example 2-1 are not necessarily coatings, and structures configured to be fixed to the wiper jaw 14 may be adopted as the pads 15 instead. A method of fixing such pads 15 to the wiper jaw 14 may be, for example: a method of making claw portions protruding from the wiper jaw 14, and mechanically fixing the pads 15 to the wiper jaw 14 by engaging the pads 15 with the claw portions; or insert molding. In this case, the plating layer 16 is not required.

Furthermore, in this modified example 2-1, a configuration without the wiper jaw 14 may be adopted as the grasper, similarly to the above described modified example 1-8. In that case, the pads 15, the electrode EP, and the first insulating portion 17 are provided on the arm 13 in an arrangement similar to the arrangement provided on the wiper jaw 14.

Modified Example 2-2

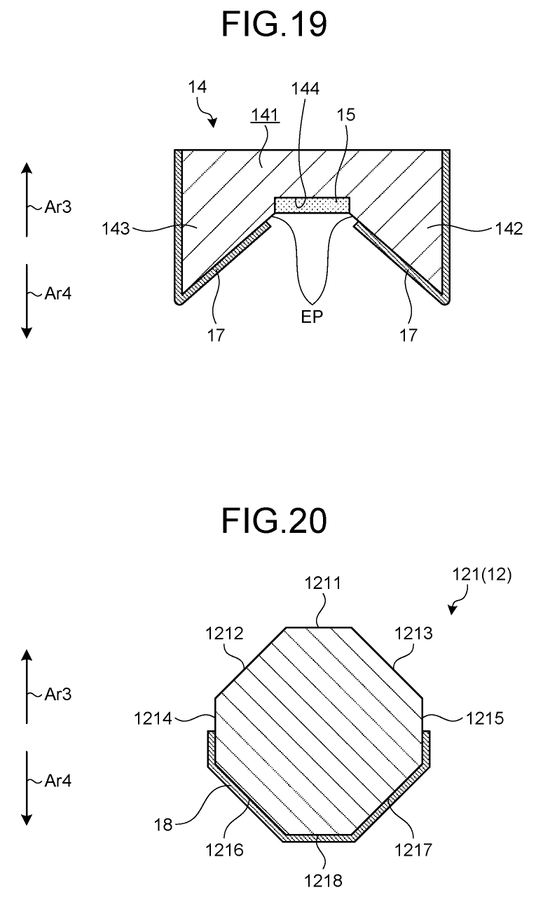

FIG. 19 is a diagram illustrating a modified example 2-2 of the second embodiment. Specifically, FIG. 19 is a diagram corresponding to FIG. 16.

In the above described second embodiment and modified example 2-1, the first insulating portion 17 may be provided in a region of surfaces of the pair of tooth portions 142 and 143, the region excluding a partial region adjacent to the center area 144, like in the modified example 2-2 illustrated in FIG. 19. That is, in this modified example 2-2, in addition to the regions of the center area 144, the regions being without the plating layer 16 and pads 15, the partial region of the surfaces of the pair of tooth portions 142 and 143 also functions as electrodes EP, the partial region being adjacent to the center area 144 and being without the first insulating portion 17.

In a case where this structure of the modified example 2-2 described above is adopted, effects similar to those of the second embodiment and modified example 2-1 described above are achieved also.

The pads 15 according to the modified example 2-2 are not necessarily coatings, and structures configured to be fixed to the wiper jaw 14 may be adopted as the pads 15 instead. A method of fixing such pads 15 to the wiper jaw 14 may be, for example: a method of making claw portions protruding from the wiper jaw 14, and mechanically fixing the pads 15 to the wiper jaw 14 by engaging the pads 15 with the claw portions; or insert molding. In this case, the plating layer 16 is not required.

Furthermore, in this modified example 2-2, a configuration without the wiper jaw 14 may be adopted as the grasper, similarly to the above described modified example 1-8. In that case, the pads 15, the electrodes EP, and the first insulating portion 17 are provided on the arm 13 in an arrangement similar to the arrangement provided on the wiper jaw 14.

Modified Example 2-3

FIG. 20 is a diagram illustrating a modified example 2-3 of the second embodiment. Specifically, FIG. 20 is a cross section of the treatment portion 121 cut along a plane orthogonal to the central axis Ax1.

In the above described second embodiment and modified examples 2-1 and 2-2, a surface of the treatment portion 121 may be covered with a second insulating portion 18 that is electrically insulating, like in the modified example 2-3 illustrated in FIG. 20. This second insulating portion 18 is a coating formed of electrically insulating rein. Examples of this resin include polyetheretherketone (PEEK).

Specifically, in this modified example 2-3, as illustrated in FIG. 20, the second insulating portion 18 is provided in a state of covering an area of a fourth surface 1214 and a fifth surface 1215, the whole sixth and seventh surfaces 1216 and 1217, and the whole eighth surface 1218, the area being approximately half the fourth and fifth surfaces 1214 and 1215 and being distant from the jaw 11.

The above described modified example 2-3 has the following effects, in addition to effects similar to those of the above described second embodiment and modified examples 2-1 and 2-2.

In a case where a treatment target is treated by application of ultrasonic vibration, temperature of a surface of the treatment portion 121 also increases, the surface being other than the first surface 1211 that is a treatment surface. In a case where that surface contacts a part other than the treatment target in body tissue in a state where the temperature of the surface has become high, an unintentional effect may be exerted on the body tissue.

In this modified example 2-3, the above described second insulating portion 18 is provided on the surface of the treatment portion 121. Therefore, any unintended effect exerted on the body tissue is able to be avoided.

The structure of this modified example 2-3 may be adopted in the configurations of the above described first embodiment and modified examples 1-6 to 1-9.

Modified Example 2-4

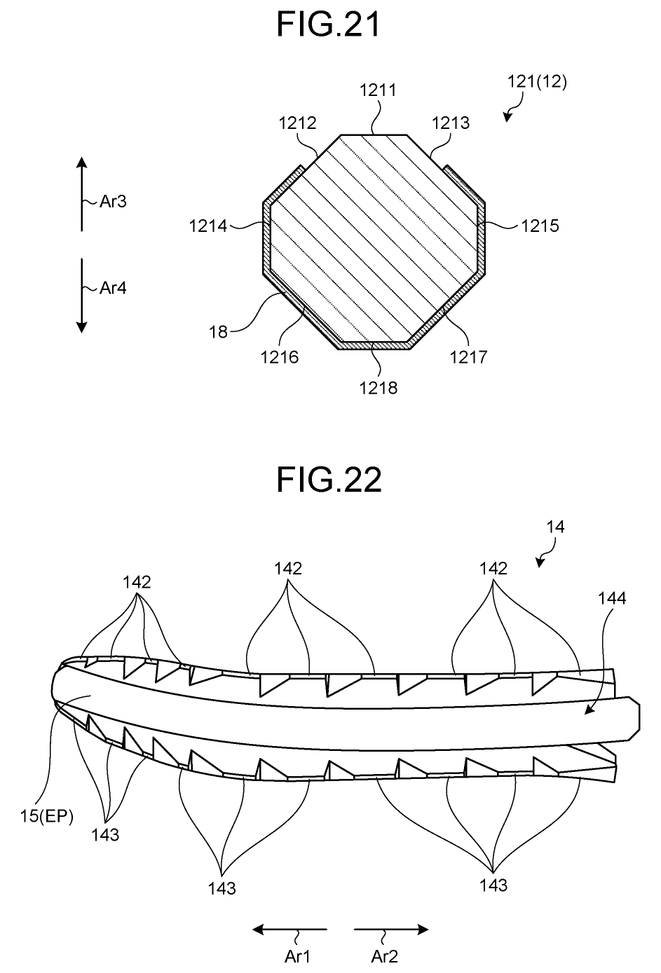

FIG. 21 is a diagram illustrating a modified example 2-4 of the second embodiment. Specifically, FIG. 21 is a diagram corresponding to FIG. 20.

The position where the second insulating portion 18 is formed may be modified in the above described modified example 2-3, like in the modified example 2-4 illustrated in FIG. 21.

Specifically, in this modified example 2-4, as illustrated in FIG. 21, the second insulating portion 18 is provided in a state of covering an area of a second surface 1212 and a third surface 1213, the whole fourth and fifth surfaces 1214 and 1215, the whole sixth and seventh surfaces 1216 and 1217, and the whole eighth surface 1218, the area being approximately half the second and third surfaces 1212 and 1213 and being distant from the jaw 11.

In a case where the structure of the modified example 2-4 described above is adopted, effects similar to those of the modified example 2-3 described above are achieved also.

The structure of this modified example 2-4 may be adopted in the configurations of the above described first embodiment and modified examples 1-6 to 1-9.

Modified Example 2-5

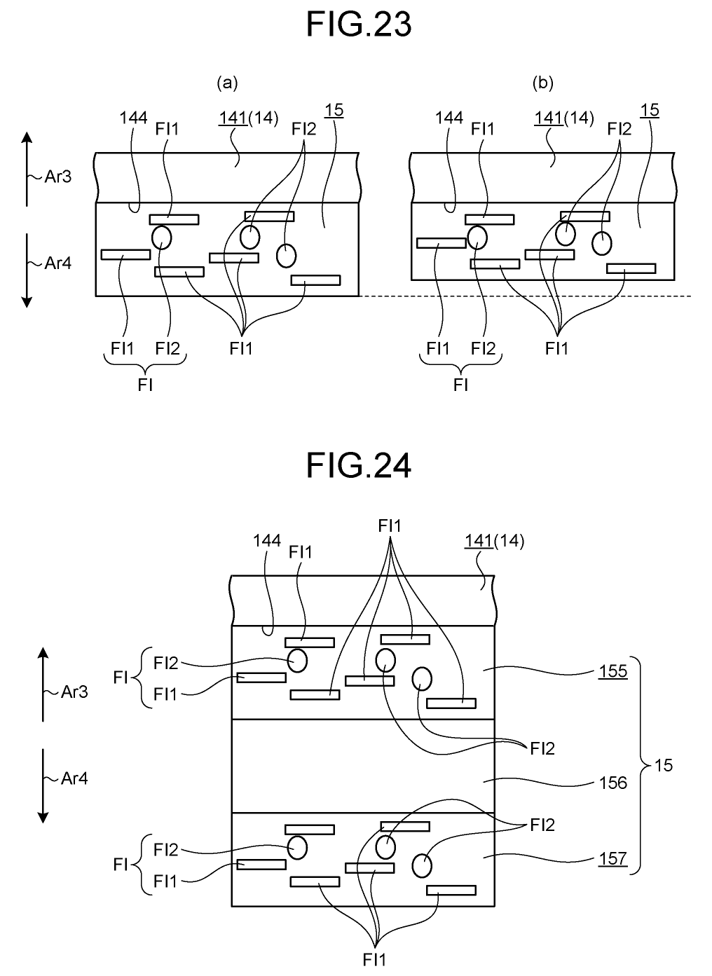

FIG. 22 to FIG. 23 are diagrams illustrating a modified example 2-5 of the second embodiment. Specifically, FIG. 22 is a diagram corresponding to FIG. 15. FIG. 23 is a cross section of a pad 15 cut along a plane orthogonal to the central axis Ax1. For convenience of explanation, illustration of the plating layer 16 has been omitted, in FIG. 23.

The configuration of the pads 15 and electrodes EP may be modified in the above described second embodiment, like the modified example 2-5 illustrated in FIG. 22 and FIG. 23.

Specifically, the pad 15 according to this modified example 2-5 is provided over the whole center area 144, similarly to the first embodiment described above, as illustrated in FIG. 22. Furthermore, electrically conducting filler FI, such as silver, carbon, or metal powder, is added in the pad 15, as illustrated in FIG. 23. This electrically conducting filler FI includes first electrically conducting filler FI1 that is scale-like and second electrically conducting filler FI2 that is granular. They are added in a state where a ratio between the amount of the first electrically conducting filler FI1 added and the amount of the second electrically conducting filler FI2 added is 2:1.

In a state where no treatment target is being grasped between the pad 15 and the treatment portion 121 and no grasping pressure is being applied to the pad 15, intervals between bits of the electrically conducting filler FI are large, as illustrated in (a) of FIG. 23. As a result, in this state, electric resistance in the pad 15 is high. That is, it is difficult for high frequency current to flow to a treatment target through the pad 15.

By contrast, in a state where a treatment target is being grasped between the pad 15 and the treatment portion 121, grasping pressure is being applied to the pad 15, temperature of the pad 15 has become high, and the pad 15 has become soft; intervals between the bits of electrically conducting filler FI are small, as illustrated in (b) of FIG. 23. As a result, in this state, electric resistance in the pad 15 is low. That is, it is easy for high frequency current to flow to the treatment target through the pad 15.

Accordingly, in this modified example 2-5, a portion of the pad 15 functions as the electrode EP (FIG. 22 and FIG. 23), the portion being where the treatment target is held between the pad 15 and the treatment portion 121 and the grasping pressure is applied.

The above described modified example 2-5 has the following effects, in addition to effects similar to those of the second embodiment described above.

The pad 15 according to this modified example 2-5 includes the electrically conducting filler FI. The portion of the pad 15 functions as the electrode EP, the portion being where the treatment target is held between the pad 15 and the treatment portion 121 and grasping pressure is applied. Therefore, application of high frequency energy is able to be concentrated on just a desired part that is being treated in the treatment target. Therefore, thermal spread is able to be reduced even further and treatment performance is able to be improved further.

The pad 15 according to the modified example 2-5 is not necessarily a coating, and a structure configured to be fixed to the wiper jaw 14 may be adopted as the pad 15 instead. A method of fixing such a pad 15 to the wiper jaw 14 may be, for example: a method of making claw portions protruding from the wiper jaw 14, and mechanically fixing the pad 15 to the wiper jaw 14 by engaging the pad 15 with the claw portions; or insert molding. In this case, the plating layer 16 is not required.

Furthermore, in this modified example 2-5, a configuration without the wiper jaw 14 may be adopted as the grasper, similarly to the above described modified example 1-8. In that case, the pad 15 and the first insulating portion 17 are provided on the arm 13 in an arrangement similar to the arrangement provided on the wiper jaw 14.

Modified Example 2-6

FIG. 24 is a diagram illustrating a modified example 2-6 of the second embodiment. Specifically, FIG. 24 is a diagram corresponding to FIG. 23.

Like in this modified example 2-6 illustrated in FIG. 24, the pad 15 in the above described modified example 2-5 may include plural (three in this modified example 2-6) layers 155 to 157 that are layered over each other. Furthermore, in this modified example 2-6, no electrically conducting filler FI is added in the layer 156 positioned between the two layers 155 and 157, as illustrated in FIG. 24.

The modified example 2-6 described above has the following effects, in addition to effects similar to those of the modified example 2-5 described above.

The pad 15 according to this modified example 2-6 includes the above mentioned plural layers 155 to 157. Therefore, electric resistance of the pad 15 is able to be set easily at a desired resistance value.

The pad 15 according to the modified example 2-6 is not necessarily a coating, and a structure configured to be fixed to the wiper jaw 14 may be adopted as the pad 15 instead. A method of fixing such a pad 15 to the wiper jaw 14 may be, for example: a method of making claw portions protruding from the wiper jaw 14, and mechanically fixing the pad 15 to the wiper jaw 14 by engaging the pad 15 with the claw portions; or insert molding. In this case, the plating layer 16 is not required.

Furthermore, in this modified example 2-6, a configuration without the wiper jaw 14 may be adopted as the grasper, similarly to the above described modified example 1-8. In that case, the pad 15 and the first insulating portion 17 are provided on the arm 13 in an arrangement similar to the arrangement provided on the wiper jaw 14.

### Other Embodiments

Modes for implementing the disclosure have been described thus far, but the disclosure is not to be limited only to the above described first and second embodiments and modified examples 1-1 to 1-9 and 2-1 to 2-6.

Modified Example 3-1

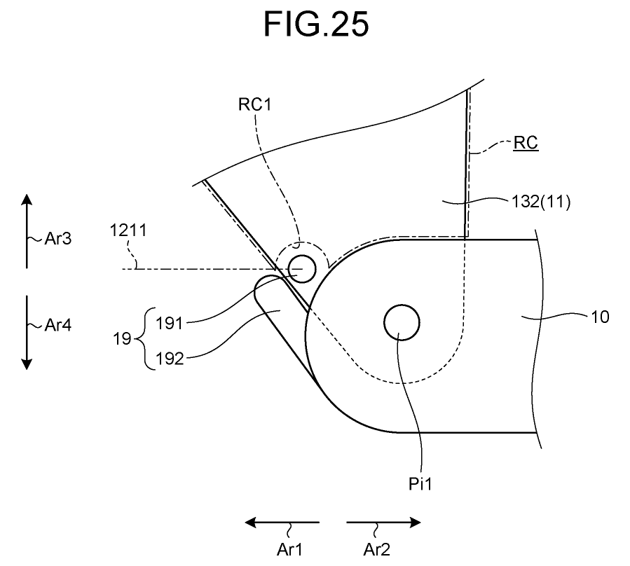

FIG. 25 is a diagram illustrating a modified example 3-1 of the first or second embodiment. Specifically, FIG. 25 is a diagram of a connection between the sheath 10 and the jaw 11, as viewed along the width direction. For convenience of explanation, FIG. 25 illustrates only the bearing portion 132 of the jaw 11. Furthermore, the cover RC is illustrated by a dashed-and-dotted line.

Like in the modified example 3-1 illustrated in FIG. 25, an interference structure 19 including a first projection portion 191 and a second projection portion 192 may be provided at the sheath 10 and the jaw 11 in the above described first and second embodiments and modified examples 1-6 to 1-9 and 2-1 to 2-6.

The first projection portion 191 corresponds to a first abutment portion. This first projection portion 191 is a projection that projects along the width direction from the bearing portion 132 of the jaw 11, as illustrated in FIG. 25. Furthermore, the first projection portion 191 projects along the width direction through a notch RC1 provided in the cover RC.

The first projection portion 191 may be provided just singly or may be provided on each of two surfaces of the bearing portion 132, the two surfaces being at ends of the width of the bearing portion 132.

The second projection portion 192 corresponds to a second abutment portion. This second projection portion 192 is a projection that projects in the distal direction Ar1 from a distal end of the sheath 10, as illustrated in FIG. 25. Furthermore, the second projection portion 192 is formed of a material having higher flexibility than the sheath 10. Furthermore, the second projection portion 192 is at a position more separate from the jaw 11 than the first surface 1211 functioning as the treatment surface is (lower in FIG. 25).

The second projection portion 192 may be provided just singly or may be provided at each of two portions of the distal end of the sheath 10, the two portions being at ends of the width of the sheath 10.

When the jaw 11 is brought close to the treatment portion 121, the first and second projection portions 191 and 192 come into contact with each other in a plane orthogonal to the rotation direction of the jaw 11 about the central axis of the first pin Pi1, at approximately the same time as a time when the pad 15 and the first surface 1211 come into contact with each other. As a result, a thermal path where heat is transmitted from the jaw 11 to the sheath 10 is formed.

The interference structure 19 is provided in the distal direction Ar1 more than the first pin Pi1 is, as illustrated in FIG. 25.

The above described modified example 3-1 has the following effects, in addition to effects similar to those of the above described first and second embodiments and modified examples 1-6 to 1-9 and 2-1 to 2-6.

In this modified example 3-1, the interference structure 19 including the first and second projection portions 191 and 192 described above is provided at the sheath 10 and jaw 11. Therefore, after treatment (incision) of a treatment target is completed, heat transmitted from the pad 15 to the jaw 11 is able to be transmitted to the sheath 10 by the interference structure 19 more. As a result, heat in the pad 15 is able to be radiated efficiently and deterioration of the pad 15 is able to be reduced further.

In particular, the interference structure 19 is provided more in the distal direction Ar1 than the first pin Pi1 is. Therefore, heat transmitted from the pad 15 to the jaw 11 is able to be transmitted to the sheath 10 at an early stage and heat in the pad 15 is able to be radiated more efficiently.

Furthermore, the second projection portion 192 is formed of a material having higher flexibility than the sheath 10. Therefore, grasping pressure applied to a treatment target by the jaw 11 and the treatment portion 121 is not changed by the interference structure 19, and the treatment target is able to be grasped at a desired grasping pressure.

Furthermore, the second projection portion 192 is at a position more separate from the jaw 11 than the first surface 1211 functioning as the treatment surface is. Therefore, the treatment surface is not restricted by the second projection portion 192 and the treatment surface is able to have a sufficient area.

Modified Example 3-2

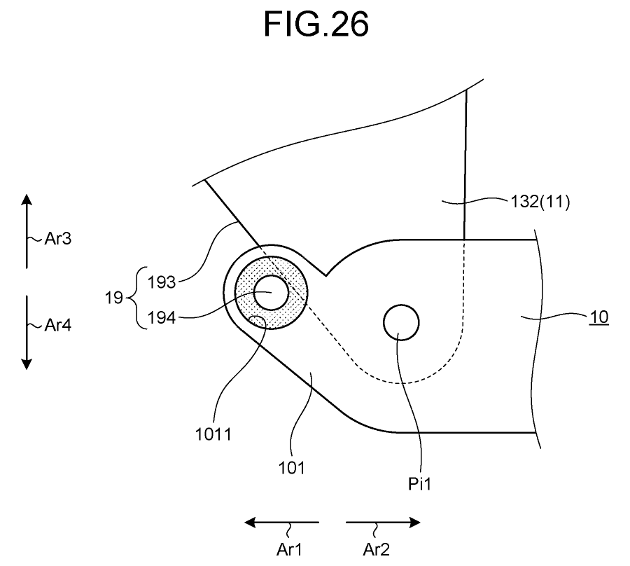

FIG. 26 is a diagram illustrating a modified example 3-2 of the first or second embodiment. Specifically, FIG. 26 is a diagram corresponding to FIG. 25. Illustration of the cover RC has been omitted in FIG. 26, in contrast to FIG. 25.

Like in this modified example 3-2 illustrated in FIG. 26, the configuration of the interference structure 19 in the above described modified example 3-1 may be modified.

The interference structure 19 according to the modified example 3-2 includes, as illustrated in FIG. 26, a first abutment portion 193 and a second abutment portion 194.

The first abutment portion 193 is a surface of the bearing portion 132 of the jaw 11, the surface being in the treatment portion direction Ar4, as illustrated in FIG. 26.

An extending portion 101 extending from the distal end of the sheath 10 in the distal direction Ar1 is provided in the sheath 10 in FIG. 26. Furthermore, a through hole 1011 penetrating the extending portion 101 along the width direction is provided in the extending portion 101.

The second abutment portion 194 is a cylindrical pin and corresponds to a second projection portion. This second abutment portion 194 is inserted through the through hole 1011 and fixed to the extending portion 101 by welding in a posture where the second abutment portion 194 is parallel to the width direction. Furthermore, the second abutment portion 194 is at a position more separate from the jaw 11 than the first surface 1211 functioning as the treatment surface is (lower in FIG. 26).

When the jaw 11 is brought close to the treatment portion 121, the first and second abutment portions 193 and 194 come into contact with each other in a plane orthogonal to the rotation direction of the jaw 11 about the central axis of the first pin Pi1, at approximately the same time as a time when the pad 15 and the first surface 1211 come into contact with each other. As a result, a thermal path where heat is transmitted from the jaw 11 to the sheath 10 is formed.

In this modified example 3-2 also, the interference structure 19 is provided more in the distal direction Ar1 than the first pin Pi1 is, as illustrated in FIG. 26.

The modified example 3-2 described above has the following effects, in addition to effects similar to those of the modified example 3-1 described above.

In this modified example 3-2, the first abutment portion 193 included in the interference structure 19 is the surface of the bearing portion 132, the surface being in the treatment portion direction Ar4. Therefore, in forming the interference structure 19, a member does not need to be provided at the jaw 11 and the interference structure 19 is able to be formed easily.

Modified Example 3-3

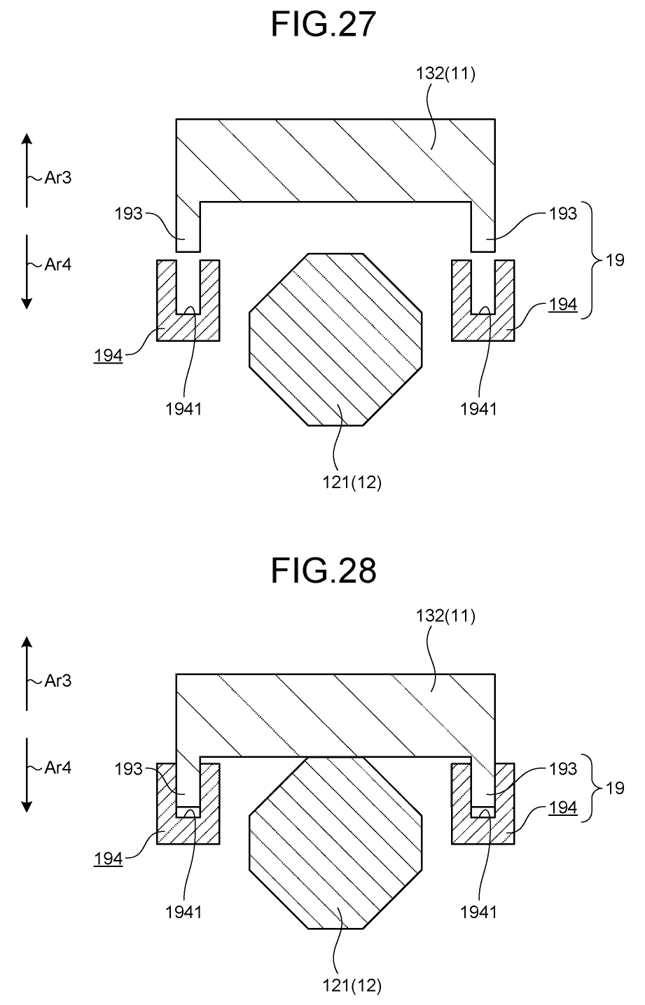

FIG. 27 and FIG. 28 are diagrams illustrating a modified example 3-3 of the first or second embodiment. Specifically, FIG. 27 and FIG. 28 are cross sections of the interference structure 19 each cut along a plane orthogonal to the central axis Ax1. FIG. 27 illustrates a state where the jaw 11 has been separated from the treatment portion 121. Furthermore, FIG. 28 illustrates a state where the jaw 11 has been brought close to the treatment portion 121.

Like in this modified example 3-3, the configuration of the interference structure 19 in the modified examples 3-1 and 3-2 described above may be modified.

The interference structure 19 according to the modified example 3-3 includes, as illustrated in FIG. 27 and FIG. 28, the first abutment portion 193 and the second abutment portion 194.

The first abutment portion 193 according to this modified example 3-3 is a projection projecting in the treatment portion direction Ar4 from the surface of the bearing portion 132 of the jaw 11, the surface being in the treatment portion direction Ar4, as illustrated in FIG. 27 and FIG. 28.

The first abutment portion 193 may be provided on each of two portions of the surface of the bearing portion 132 as illustrated in FIG. 27 and FIG. 28, the surface being in the treatment portion direction Ar4, the two portions being at both ends of the width of the surface, or may be provided just singly.

The second abutment portion 194 according to the modified example 3-3 is a portion extending in the distal direction Ar1 from the distal end of the sheath 10. Furthermore, a recessed portion 1941 is provided on a surface of the second abutment portion 194, the surface being near the jaw 11, as illustrated in FIG. 27 and FIG. 28. Furthermore, the second abutment portion 194 is at a position more separate from the jaw 11 than the first surface 1211 functioning as the treatment surface is (lower in FIG. 27 and FIG. 28).

The second abutment portion 194 may be provided on each of two portions of the distal end of the sheath 10 as illustrated in FIG. 27 and FIG. 28, the two portions being at ends of the width of the distal end, or may be provided just singly.

When the jaw 11 is brought close to the treatment portion 121, the first abutment portion 193 is inserted in the recessed portion 1941, and the first and second abutment portions 193 and 194 come into contact with each other in planes parallel to the rotation direction (a vertical direction in FIG. 27 and FIG. 28) of the jaw 11 about the central axis of the first pin Pi1. As a result, thermal paths where heat is transmitted from the jaw 11 to the sheath 10 are formed.

In this modified example 3-3 also, the interference structure 19 is provided more in the distal direction Ar1 than the first pin Pi1 is.

The modified example 3-3 described above has the following effects, in addition to effects similar to those of the modified examples 3-1 and 3-2 described above.

When the jaw 11 is brought close to the treatment portion 121, the first and second abutment portions 193 and 194 of the interference structure 19 according to the modified example 3-3 come into contact with each other in the planes parallel to the rotation direction of the jaw 11 about the central axis of the first pin Pi1. Therefore, the contact area where the first and second abutment portions 193 and 194 contact each other is able to be increased and heat in the pad 15 is able to be radiated more efficiently.

Modified Example 3-4

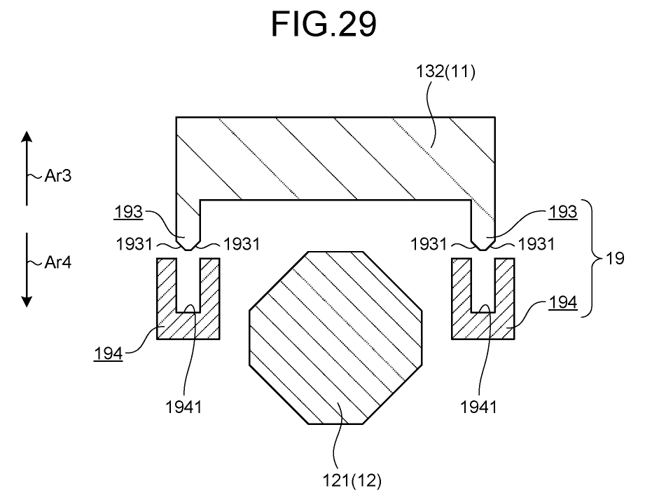

FIG. 29 is a diagram illustrating a modified example 3-4 of the first or second embodiment. Specifically, FIG. 29 is a diagram corresponding to FIG. 27.

Like in this modified example 3-4 illustrated in FIG. 29, inclined surfaces 1931 may be provided at a tip of the first abutment portion 193 in the above described modified example 3-3.

The area of the cross section of the first abutment portion 193 according to the modified example 3-4 decreases toward the tip, due to these inclined surfaces 1931. The inclined surfaces 1931 guide insertion of the first abutment portion 193 into the recessed portion 1941.

Instead of providing the inclined surfaces 1931 in the first abutment portion 193, inclined surfaces having the same function as the inclined surfaces 1931 may be provided at an opening portion of the recessed portion 1941 so that the area of the opening of the recessed portion 1941 increases toward the jaw 11. Furthermore, these inclined surfaces may be provided in the opening portion of the recessed portion 1941, in addition to the inclined surfaces 1931 provided in the first abutment portion 193.

The modified example 3-4 described above has the following effects, in addition to effects similar to those of the modified example 3-3 described above.

In this modified example 3-4, the inclined surfaces 1931 are provided in the first abutment portion 193. Therefore, the insertion of the first abutment portion 193 into the recessed portion 1941 is guided by the inclined surfaces 1931 and the first abutment portion 193 is thus able to be inserted into the recessed portion 1941 smoothly.

Modified Example 3-5

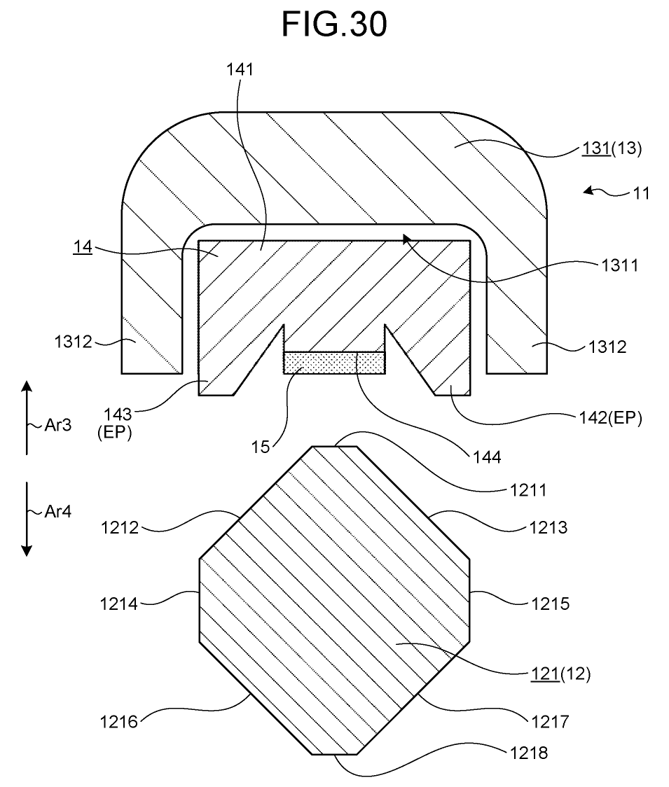

FIG. 30 is a diagram illustrating a modified example 3-5 of the first or second embodiment. Specifically, FIG. 30 is a diagram corresponding to FIG. 3.

In the above described first and second embodiments and modified examples 1-6 to 1-9 and 2-1 to 2-6, the pad 15 may be directly provided in the wiper jaw 14 or the arm 13, without provision of the plating layer 16. FIG. 30 illustrates, as an example, a configuration having the pad 15 directly provided on the wiper jaw 14 in the first embodiment described above.

In a case where the above described structure of the modified example 3-5 is adopted, effects similar to those of the above described first and second embodiments and modified examples 1-6 to 1-9 and 2-1 to 2-6 are achieved also.

Modified Example 3-6

In the above described first and second embodiments and modified examples 1-6 to 1-9 and 2-1 to 2-6, the resin forming the pad 15 is not necessarily PTFE, and other resin, for example, other fluororesin, may be adopted instead.

In a case where the above described structure of the modified example 3-6 is adopted, effects similar to those of the above described first and second embodiments and modified examples 1-6 to 1-9 and 2-1 to 2-6 are achieved also.

The following configurations also belong to the technical scope of the present disclosure.

(1) An ultrasonic treatment tool, comprising:

a vibration transmission member including a treatment portion at a distal end of the vibration transmission member, the treatment portion being configured to treat a body tissue, the vibration transmission member configured to transmit ultrasonic vibration from a proximal end of the vibration transmission member to the treatment portion; a sheath where the vibration transmission member is inserted through, with the treatment portion protruding from the sheath; and a grasper that is rotatably connected to the sheath, the grasper being configured to be opened or closed relatively to the treatment portion by rotating relatively to the sheath and grasp the body tissue between the grasper and the treatment portion, wherein an interference structure is provided at the sheath and the grasper, the interference structure forming a thermal path to transmit heat from the grasper to the sheath when the grasper is closed relatively to the treatment portion.

(2) The ultrasonic treatment tool according to (1) above, wherein the interference structure includes a first projection portion provided in the grasper.

(3) The ultrasonic treatment tool according to (2) above, wherein the interference structure includes a second projection portion that is provided in the sheath and is where the first projection portion is in contact with when the grasper is closed relatively to the treatment portion.

(4) The ultrasonic treatment tool according to (1) above, wherein the interference structure is provided more in a distal direction than a connection position is, the connection position being where the grasper is rotatably connected to the sheath.

(5) The ultrasonic treatment tool according to (3) above, wherein the second projection portion has higher flexibility than the sheath.

(6) The ultrasonic treatment tool according to (1) above, wherein the interference structure includes a second projection portion provided in the sheath.

(7) The ultrasonic treatment tool according to (1) above, wherein the interference structure includes a first abutment portion and a second abutment portion configured to be in contact with each other when the grasper is closed relatively to the treatment portion, and the first abutment portion and the second abutment portion are in contact with each other in a plane orthogonal to a direction of rotation of the grasper relative to the sheath.

(8) The ultrasonic treatment tool according to (1) above, wherein the interference structure includes a first abutment portion and a second abutment portion configured to be in contact with each other when the grasper is closed relatively to the treatment portion, and the second abutment portion is provided in the sheath and is at a position more separate from the grasper than a treatment surface of the treatment portion is.

(9) The ultrasonic treatment tool according to (1) above, wherein the interference structure includes a first abutment portion and a second abutment portion configured to be in contact with each other when the grasper is closed relatively to the treatment portion, and the first abutment portion and the second abutment portion are in contact with each other in a plane parallel to a direction of rotation of the grasper relative to the sheath.

(10) The ultrasonic treatment tool according to (9) above, wherein a recessed portion is provided in one abutment portion of the first abutment portion and the second abutment portion, and the other abutment portion of the first abutment portion and the second abutment portion is inserted into the recessed portion when the grasper is closed relatively to the treatment portion.

(11) The ultrasonic treatment tool according to (10) above, wherein an inclined surface that guides insertion of the other abutment portion into the recessed portion is provided in at least one of the first abutment portion and the second abutment portion.

(12) The ultrasonic treatment tool according to (1) above, wherein a cover is attached to the grasper, and the interference structure forms the thermal path through a through hole or notch provided in the cover. The disclosure enables provision of an ultrasonic treatment tool including an improved pad. Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic treatment tool, comprising:
a blade including a treatment portion at a distal side of the blade, the blade configured to transmit an ultrasonic vibration from a proximal side of the blade to the treatment portion and the treatment portion configured to treat a body tissue; and
a grasper movable relative to the treatment portion between an open position and a closed position to grasp the body tissue between the grasper and the treatment portion,
wherein the grasper includes:
a plurality of pads extending in a longitudinal direction of the grasper, the plurality of pads contacting the treatment portion when the grasper is in the closed position,
an electrode between the plurality of pads and configured to flow a high frequency current between the electrode and the treatment portion when the grasper is in the closed position, and
a pair of insulation members extending across the plurality of pads and the electrode.

2. The ultrasonic treatment tool according to claim 1, wherein the electrode comprises a plurality of electrodes.

3. The ultrasonic treatment tool according to claim 1, wherein a periphery of the electrode is surrounded by the plurality of pads.

4. The ultrasonic treatment tool according to claim 1, wherein a periphery of the plurality of the pads is surrounded by the electrode.

5. The ultrasonic treatment tool according to claim 1, wherein a part of the treatment portion is covered with a coating that includes an electrically insulating resin.

6. The ultrasonic treatment tool according to claim 1, wherein each of the plurality of pads has a coating including a resin.
